SEQUENCE LISTING (4 sequences)

SEQ ID NO: 1 - DNA, 20 bp, Artificial sequence
  Synthetic oligonucleotide
tccatgacgttcctgatgct

SEQ ID NO: 2 - DNA, 20 bp, Artificial sequence
  Synthetic oligonucleotide
tccatgagcttcctgatgct

SEQ ID NO: 3 - DNA, 20 bp, Artificial sequence
  Synthetic oligonucleotide
tccatgacgttcctgggggg

SEQ ID NO: 4 - protein, 8 aa, Artificial sequence
  Synthetic oligopeptide
SIINFEKL

(19) United States
(12) Patent Application Publication
(10) Pub. No.: US 2008/0113929 A1
(43) Pub. Date: May 15, 2008

(54) Abasic Oligonucleotide as Carrier Platform for Antigen and Immunostimulatory Agonist and Antagonist

(75) Inventors: Grayson B. Lipford, Watertown, MA (US); Alexandra Forsbach, Ratingen (DE); Eugen Uhlmann, Glashuetten (DE); Hermann Wagner, Eching (DE)

Correspondence Address: WOLF GREENFIELD & SACKS, P.C., 600 ATLANTIC AVENUE, BOSTON, MA, 02210-2206, US

(73) Assignee: Coley Pharmaceutical GmbH, Langenfeld (DE)

(21) Appl. No.: 11/629,106

(22) PCT Filed: Jun. 8, 2005
(86) PCT No.: PCT/US05/20225

§ 371 Date: Aug. 27, 2007

Related U.S. Application Data
(60) Provisional application No. 60/577,813, filed on Jun. 8, 2004.

(51) Int. Cl. A61K 48/00

(52) U.S. Cl. 514/44; 514/81

(57) ABSTRACT

Compositions and methods are provided for enhancing delivery of therapeutic agents. More specifically, compositions and methods are provided for improving antigen delivery to antigen-presenting cells. Conjugates between abasic oligonucleotides and antigen are provided, along with methods for their use in vaccination and in the treatment of cancer, infection, and allergy and asthma. Also provided are conjugates between abasic oligonucleotides and various immunostimulatory nucleic acids, including CpG oligonucleotides, as well as methods of use thereof. Also provided are conjugates between abasic oligonucleotides and various other agonists and antagonists of immunostimulation, as well as methods of use thereof.

### BACKGROUND OF THE INVENTION

Immunostimulatory nucleic acids including CpG DNA have recently been described to be potent adjuvants. Immunostimulatory CpG DNA activates immature dendritic cells (DC) via Toll-like receptor 9 (TLR9). Extensive study has led to an appreciation that the efficacy of immunostimulatory nucleic acids, including CpG DNA, is sequence-dependent. For example, potent immunostimulatory DNA molecules can be rendered essentially inactive simply by reversing CpG dinucleotides to GpC dinucleotides. In addition, the sequence context surrounding an unmethylated CpG dinucleotide can dramatically influence the immunostimulatory potential of a CpG nucleic acid. Lipford G B et al. (1997) Eur J Immunol 27:3420-6; Sparwasser T et al. (2000) Eur J Immunol 30:3591-7; Hemmi H et al. (2000) Nature 408:740-5; Bauer S et al. (2001) Proc Natl Acad Sci USA 98:9237-42.

As part of an effort to define the sequence specificity of the immunostimulatory effect of CpG DNA, others have examined not only the role of specific nucleobases in specific positions flanking the CpG dinucleotide, but also substitution of such nucleobases with abasic nucleosides, i.e., with 1′,2′-dideoxynucleosides. Yu D et al. (2001) Bioorg Med Chem Lett 11:2263-7; Agrawal S et al. (2002) Trends Mol Med 8:114-21. Deletion of one or two nucleobases in the 3′-flanking sequence three or more nucleosides from a CpG dinucleotide was reported to have little or no effect on immunostimulatory activity, while similar substitutions in the 5′-flanking sequence reportedly increased immunostimulatory activity. Ibid.

Despite this appreciation of sequence specificity for immunostimulatory nucleic acids, details of the mechanisms through which they exert their immunostimulatory effects remain to be elucidated. It is not yet known, for example, how CpG DNA interacts with TLR9, or exactly how CpG DNA is internalized into a cell to interact with TLR9 which resides in late (Lamp1+) endosomal organelles. Wagner H. (2001) Immunity 14:499-502; Ahmad-Nejad P et al. (2002) Eur J Immunol 32:1958-68. It is believed that there is some differentially expressed cell surface receptor, yet to be defined, that is involved in nucleic acid uptake. This receptor appears to be expressed preferentially on antigen-presenting cells, i.e., DC, macrophages, monocytes, and B cells, and not on T cells.

Dendritic cells are crucial for the initiation of primary T-cell responses. Immature DC lack costimulatory signals required for productive T-cell activation but are well equipped to sample antigen. Antigen sampling can be accomplished through fluid phase pinocytosis or by relatively more efficient receptor-mediated endocytosis. Following DC maturation, antigen sampling ceases, expression of costimulatory molecules and MHC-peptide complexes increases, and Th1-promoting cytokines are produced. Banchereau J et al. (1998) Nature 392:245-52.

Crosslinking of immunostimulatory DNA sequences with proteinaceous antigen results in cytotoxic T lymphocyte (CTL) priming and Th1-biased immune responses, as reported by Cho and colleagues. Cho H J et al. (2000) Nat Biotechnol 18:509-14. Using phycobiliprotein-CpG-DNA conjugates, Shirota and colleagues reported DNA-guided augmentation of antigen sampling by DC. Shirota H et al. (2001) J Immunol 167:66-74. The instant inventors previously reported that conjugates of CpG DNA and peptide antigen can shift antigen uptake by immature DC from rather inefficient fluid phase pinocytosis to more efficient receptor-mediated endocytosis. Maurer T et al. (2002) Eur J Immunol 32:2356-64. Cellular uptake of antigen was equally enhanced for conjugates regardless of DNA sequence, while DC maturation required immunostimulatory CpG sequence. Ibid.

### SUMMARY OF THE INVENTION

The invention is based in part on the discovery by the inventors that an abasic oligonucleotide is an effective carrier for the delivery of agents to cells capable of taking up nucleic acid molecules. As disclosed herein, the invention uses abasic oligonucleotide as a mimic of DNA or RNA to utilize receptor-driven uptake into cells of antigen or drug, wherein the antigen or drug is provided as a conjugate with abasic oligonucleotide. The invention thus is useful whenever it is desired to deliver a compound to the interior of a cell that is capable of taking up nucleic acid molecules. In particular the invention is useful for improved delivery of antigens to antigen-presenting cells. The invention is also particularly useful for delivery of immunostimulatory ligands and other molecules to cells of the immune system. The invention encompasses both compositions and methods of use of the compositions, both in vitro and in vivo.

In one aspect the invention provides a conjugate including an abasic oligonucleotide 10-40 units long and a therapeutic agent. As further disclosed below, an abasic oligonucleotide resembles a backbone of a DNA or an RNA molecule, wherein the nucleobases (e.g., adenine, cytosine, thymine, uracil, and guanine) and optionally the sugar residues are absent. The abasic oligonucleotide is thus a polymer of units connected by phosphate-containing linkages. Each unit of the polymeric abasic oligonucleotide includes a phosphate group, or a thioated derivative thereof, covalently linked to an organic residue which contains at least three carbon atoms. The organic residue comprises an alkyl group, either linear or cyclic, being saturated or unsaturated, which can contain O, N and S heteroatoms, and in addition can include substituents containing C, H, N, O, S, halogen atoms, and any combination thereof.

The organic residue is preferably derived from propane-1,3-diol or sugar residues, such as β-D-deoxyribofuranose or β-D-ribofuranose. Other residues include butane-1,4-diol, triethylene glycol units, or hexaethylene glycol units ((OCH₂CH₂)ₚO, where p is 3 or 6), hydroxyl-alkyl-amino linkers, such as C3, C6, C12 aminolinkers, and also alkylthiol linkers, such as C3 or C6 thiol linkers. The sugar derivatives can also contain ring expansions, such as pyranose.

The abasic oligonucleotide can also contain a Doubler or Trebler unit (Glen Research, Sterling, Va.), in particular comprising a 3′3′-linkage. Branching of the oligonucleotides by multiple doubler, trebler, or other multiplier units leads to dendrimers which are a further embodiment of this invention.

In one embodiment a unit can be an abasic deoxyribonucleotide represented as

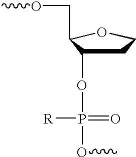

wherein R represents oxygen, sulfur, methyl, or O-alkyl.

In one embodiment a unit can be an abasic ribonucleotide represented as

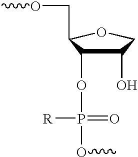

wherein R represents oxygen, sulfur, methyl, or O-alkyl.

In one embodiment a unit can be a C3 spacer/phosphate represented as

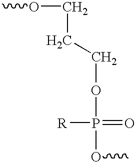

wherein R represents oxygen, sulfur, methyl, or O-alkyl.

In one embodiment the abasic oligonucleotide is a homopolymer of abasic deoxyribonucleotides (poly-D). Each unit in this embodiment includes an abasic 2′-deoxyribose sugar residue and a 5′ phosphate group. In another embodiment the abasic oligonucleotide is a homopolymer of abasic ribonucleotides. Each unit in this embodiment includes an abasic 2′-hydroxyribose sugar residue and a 5′ phosphate group.

In another embodiment the abasic oligonucleotide is a heteropolymer of abasic ribonucleotides and abasic deoxyribonucleotides. The abasic ribonucleotides and abasic deoxyribonucleotides in this embodiment can be present in any integer ratio, e.g., 19:1, referring to 19 abasic ribonucleotides to every one abasic deoxyribonucleotide. The ratio can range from 1:9 to 9:1 for an abasic oligonucleotide that is 10 units long. The ratio can range from 1:39 to 39:1 for an abasic oligonucleotide that is 40 units long. Ratios can similarly range from 1:(n−1) to (n−1): 1 for any abasic oligonucleotide that is n units long.

The abasic oligonucleotide need not include a sugar residue but can instead include just the three-carbon structure from the sugar that corresponds to the 3′, 4′, and 5′ positions of the sugar. Thus in one embodiment the abasic oligonucleotide is a homopolymer of C3 spacers derived from propane-1,3-diol.

In one embodiment the units of the abasic oligonucleotide are linked by phosphodiester linkages. In one embodiment the units of the abasic oligonucleotide are linked by phosphorothioate linkages. In one embodiment the units of the abasic oligonucleotide are linked by a combination of phosphodiester linkages and phosphorothioate linkages.

In various individual embodiments the abasic oligonucleotide according to this and other aspects of the invention is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 units long.

Also according to this and other aspects of the invention, in one embodiment the therapeutic agent is an antigen. The antigen according to this and other aspects of the invention can, in various embodiments, be an antigen characteristic of an infectious agent, an antigen characteristic of a cancer, an antigen characteristic of an autoimmune disease, an alloantigen, or an allergen.

In one embodiment the therapeutic agent is an immunostimulatory nucleic acid molecule. In one embodiment the immunostimulatory nucleic acid molecule is a CpG nucleic acid molecule. In a particular embodiment the immunostimulatory nucleic acid molecule is a CpG oligonucleotide.

In one embodiment the therapeutic agent is a small molecule. In one embodiment the small molecule is a Toll-like receptor (TLR) signaling agonist. In another embodiment the small molecule is a TLR signaling antagonist.

The conjugate according to this and other aspects of the invention can include more than one abasic oligonucleotide, more than one therapeutic agent, or more than one abasic oligonucleotide and more than one therapeutic agent. In one embodiment the therapeutic agent is a plurality of identical therapeutic agents. In another embodiment the therapeutic agent includes a plurality of non-identical therapeutic agents.

When the conjugate includes a single therapeutic agent, the single therapeutic agent can be linked to a single unit of the abasic oligonucleotide. Alternatively, when the conjugate includes a single therapeutic agent, the single therapeutic agent can be linked to more than a single unit of the abasic oligonucleotide.

When the conjugate includes a plurality of therapeutic agents, identical or otherwise, one or more therapeutic agents can be linked to one or more units of the abasic oligonucleotide. In one embodiment a plurality of therapeutic agents is linked to a single unit of the abasic oligonucleotide. In one embodiment each and every unit is linked to at least one therapeutic agent. In one embodiment each and every unit is linked to one therapeutic agent. In one embodiment at least one unit is linked to at least one therapeutic agent and at least one unit is not linked to any therapeutic agent.

In one embodiment according to this aspect of the invention, the abasic oligonucleotide and the therapeutic agent are covalently coupled.

In one embodiment the abasic oligonucleotide includes a 5′ end and a 3′ end, and the therapeutic agent is covalently coupled to the 3′ end of the abasic oligonucleotide. In another embodiment, the abasic oligonucleotide includes a 5′ end and a 3′ end and the therapeutic agent is covalently coupled to the 5′ end of the abasic oligonucleotide. In yet another embodiment, the conjugate includes a first abasic oligonucleotide having a first 5′ end and first 3′ end, a second abasic oligonucleotide having a second 5′ end and a second 3′ end, and a therapeutic agent, wherein the therapeutic agent is covalently coupled to the first 3′ end of the first abasic oligonucleotide and is also covalently coupled to the second 5′ end of the second abasic oligonucleotide. In yet another embodiment, the two abasic oligonucleotides are connected to the therapeutic agent via the two 3′ ends while the 5′ ends are free. In yet another embodiment, the two abasic oligonucleotides are connected to the therapeutic agent via the two 5′ ends while the 3′ ends are free.

In each of the foregoing embodiments, the abasic oligonucleotide and the therapeutic agent can be covalently coupled through a linker. In one embodiment the linker is susceptible to cleavage by an enzyme.

In one embodiment the abasic oligonucleotide is at least 20 units long.

In one embodiment the abasic oligonucleotide is 20 units long.

In one embodiment the conjugate is a pharmaceutical composition that further includes a pharmaceutically acceptable carrier. The conjugate of the pharmaceutical composition can include or be in the form of a pharmaceutically acceptable salt or hydrate of the conjugate. The invention also provides a method for making a pharmaceutical composition of the invention. The method includes the step of placing a therapeutically effective amount of a conjugate of the invention, or a pharmaceutically acceptable salt or hydrate thereof, in a pharmaceutically acceptable carrier.

In one aspect the invention provides a composition including a conjugate of at least one abasic oligonucleotide and an immunostimulatory nucleic acid molecule, wherein the conjugate includes at least 4 abasic units and the immunostimulatory nucleic acid includes at least 6 nucleotides, such that the conjugate is 10-40 units and nucleotides long. In various individual embodiments the conjugate according to this aspect of the invention is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 units and nucleotides long. In one embodiment the abasic oligonucleotide is 5′ to the immunostimulatory nucleic acid molecule. In one embodiment the abasic oligonucleotide is 3′ to the immunostimulatory nucleic acid molecule. In one embodiment the immunostimulatory nucleic acid molecule is flanked by a 5′ abasic oligonucleotide and by a 3′ abasic oligonucleotide, wherein each of the 5′ abasic oligonucleotide and the 3′ abasic oligonucleotide is independently at least one unit long. In the latter embodiment the 5′ flanking abasic oligonucleotide and the 3′ flanking abasic oligonucleotide can be of the same or different lengths, provided there are at least 4 abasic units in total in the conjugate. Also according to this latter embodiment, the 5′ flanking abasic oligonucleotide and the 3′ flanking abasic oligonucleotide can be of the same or different composition with respect to the type or types of abasic units within each flanking abasic oligonucleotide. In various individual embodiments the conjugate according to this aspect of the invention includes a total of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 abasic units.

In one embodiment the immunostimulatory nucleic acid molecule is a CpG oligonucleotide having at least the following structure: X₁X₂CGX₃X₄, wherein C is unmethylated cytidine, G is guanosine, and X₁, X₂, X₃, and X₄ are nucleotides.

In one aspect the invention provides use of a composition of the invention for manufacture of a medicament useful in treating an infection in a subject. In one embodiment the composition includes a conjugate of an abasic oligonucleotide 10-40 units long and a therapeutic agent.

In one aspect the invention provides use of a composition of the invention for manufacture of a medicament useful in treating an allergic condition in a subject. In one embodiment the composition includes a conjugate of an abasic oligonucleotide 10-40 units long and a therapeutic agent. In one embodiment according to this aspect of the invention, the allergic condition is allergic asthma.

In one aspect the invention provides use of a composition of the invention for manufacture of a medicament useful in treating a cancer in a subject. In one embodiment the composition includes a conjugate of an abasic oligonucleotide 10-40 units long and a therapeutic agent.

In one aspect the invention provides use of a composition of the invention for manufacture of a medicament useful in treating an autoimmune disease in a subject. In one embodiment the composition includes a conjugate of an abasic oligonucleotide 10-40 units long and a therapeutic agent.

In one aspect the invention provides use of a composition of the invention for manufacture of a medicament useful in treating an inflammatory response in a subject. In one embodiment the composition includes a conjugate of an abasic oligonucleotide 10-40 units long and a therapeutic agent.

In one aspect the invention provides use of a composition of the invention for manufacture of a medicament useful in vaccinating a subject against the antigen. In one embodiment the composition includes a conjugate of an abasic oligonucleotide 10-40 units long and an antigen.

In one aspect the invention provides a vaccine including an abasic oligonucleotide 10-40 units long covalently linked to an antigen. The antigen according to this and other aspects of the invention can, in various embodiments, be an antigen characteristic of an infectious agent, an antigen characteristic of a cancer, an antigen characteristic of an autoimmune disease, an alloantigen, or an allergen. In one embodiment according to this and other aspects of the invention the antigen is an antigen per se.

Also provided in one aspect of the invention is a method of increasing antigen uptake by an antigen-presenting cell (APC). The method according to this aspect of the invention includes the step of contacting an APC with a composition of the invention in an effective amount to permit antigen uptake by the APC, wherein for a given amount of the antigen, an amount of the antigen taken up by the APC is greater when the APC is contacted with the conjugate than when the APC is contacted with the antigen alone. In one embodiment the composition of the invention includes a conjugate of an abasic oligonucleotide 10-40 units long and an antigen.

In one embodiment the antigen includes a polypeptide.

In one embodiment the contacting occurs in vivo.

The invention further provides, according to one aspect, a method of vaccinating a subject. The method according to this aspect of the invention involves the step of administering to a subject a composition of the invention in an effective amount to induce an antigen-specific immune response to the antigen in the subject. In one embodiment the composition of the invention includes a conjugate of an abasic oligonucleotide 10-40 units long and an antigen.

In yet a further aspect the invention provides a method of increasing delivery of a TLR signaling agonist to a TLR. The method according to this aspect of the invention includes the step of contacting a cell expressing a TLR with a composition of the invention in an effective amount to deliver the TLR signaling agonist to the TLR, wherein for a given amount of the TLR signaling agonist, an amount of the TLR signaling agonist delivered to the TLR is greater when the cell is contacted with the conjugate than when the cell is contacted with the TLR signaling agonist alone. In one embodiment the composition of the invention includes a conjugate of an abasic oligonucleotide 10-40 units long and a signaling agonist specific for the TLR.

In one embodiment the TLR is TLR9. In another embodiment the TLR is TLR8. In yet another embodiment the TLR is TLR7. In yet another embodiment the TLR is TLR3.

In one embodiment the TLR signaling agonist is a CpG oligonucleotide.

In one embodiment the TLR signaling agonist is a small molecule.

In one embodiment the TLR signaling agonist is an RNA molecule.

In one embodiment the TLR signaling agonist is a double-stranded RNA.

In one embodiment the contacting occurs in vivo.

### BRIEF DESCRIPTION OF THE DRAWINGS

FIG. 1 is a bar graph depicting uptake by RAW 264.7 cells of Cy3-labeled 20-mer abasic oligonucleotides (poly-D or poly-C3) and of 20-mer ODN 5890 (SEQ ID NO:5). No data is shown for poly-C3 at 4.0 or 5.0 μM.

FIG. 2 is a graph depicting fold induction of TLR9 signaling in vitro, as measured using 293 cells stably transformed with murine TLR9 and a NF-κB-luciferase reporter construct, following 16 hour incubation with indicated concentrations of hexamer CpG motif GACGTT alone (20321), CpG motif GACGTT in the context of a 20-mer CpG-ODN(ODN 5890; SEQ ID NO:5), or CpG motif GACGTT in the context of flanking abasic sequences in 20-mer 20307 (poly-D) or in 20-mer 20566 (poly-C3 spacer). EC₅₀ values are shown in the graph legend.

### DETAILED DESCRIPTION OF THE INVENTION

It has been appreciated for some time that certain nucleic acid molecules, notably oligonucleotides, can be taken up by cells and stimulate an immune response. The precise mechanism by which nucleic acid molecules are taken up by cells is not known. However, a number of studies have concluded that uptake is possibly affected by backbone composition and by base composition. More specifically, it has been reported that phosphorothioate backbone oligonucleotides may be taken up preferentially over phosphodiester backbone oligonucleotides. It has also been reported that oligonucleotides containing poly-G sequences, i.e., oligonucleotides containing four or more consecutive guanosine nucleotides, are preferentially taken up by cells in favor of random sequence. Aside from poly-G, it appears that nucleic acid uptake by cells is essentially sequence-nonspecific.

While the identity of the nucleic acid transporter remains unknown, it appears to have a restricted expression. For example, nucleic acid uptake appears to be relatively efficient in professional antigen-presenting cells (APC), including dendritic cells (myeloid and lymphoid), macrophages, monocytes, and B lymphocytes (B cells). In contrast, T lymphocytes (T cells) appear to have relatively poor uptake of nucleic acids.

Interest in nucleic acids as therapeutic agents has been heightened by the recent appreciation of certain base sequence-specific effects of nucleic acids, including their use as antisense, small interfering RNA (siRNA), ribozyme, immunostimulatory, immunoinhibitory, and gene replacement agents. For example, there has been a great deal of effort directed toward understanding the mechanism of action of immunostimulatory CpG nucleic acids.

The instant invention is based in part upon the discovery by the inventors that cells of the immune system efficiently take up abasic oligonucleotides and that such oligonucleotides can be conjugated to therapeutics in order to improve and to direct delivery of the therapeutics to cells expressing nucleic acid transporters. The invention is useful in a number of applications, including vaccination, regulating and shaping an immune response, drug delivery in general, and treating a variety of diseases and conditions including, without limitation, infection, inflammation, allergy, cancer, transplantation, and autoimmunity.

### DEFINITIONS

As used herein, an “abasic oligonucleotide” refers to an oligomer 2-200 units long containing covalently linked units chosen from abasic deoxyribonucleotides, abasic ribonucleotides, C3 spacers, and any combination thereof. An abasic oligonucleotide can have a 5′ end, a 3′ end, or both a 5′ end and a 3′ end. In embodiments involving abasic oligonucleotides having one or more C3 spacer units, an abasic oligonucleotide can have an end corresponding to a 5′ end, an end corresponding to 3′ end, or both an end corresponding to a 5′ end and an end corresponding to 3′ end. As used hereinbelow, an end corresponding to a 5′ end shall be referred to as a 5′ end, and an end corresponding to 3′ end shall be referred to as a 3′ end.

As used herein, an “abasic deoxyribonucleotide” refers to a 2-deoxyribose sugar-phosphate moiety which resembles a unit of a DNA polymer without the nucleobase (e.g., adenine, cytosine, guanine, thymine, or uracil). Abasic deoxyribonucleotides can be linked together through their phosphate groups to form abasic oligonucleotides. Abasic deoxyribonucleotides can also be linked together with abasic ribonucleotides and/or C3 spacers through their phosphate groups to form abasic oligonucleotides.

As used herein, an “abasic ribonucleotide” refers to a 2-hydroxyribose sugar-phosphate moiety which resembles a unit of an RNA polymer without the nucleobase (e.g., adenine, cytosine, guanine, thymine, or uracil). Abasic ribonucleotides can be linked together through their phosphate groups to form abasic oligonucleotides. Abasic ribonucleotides can also be linked together with abasic deoxyribonucleotides and/or C3 spacers through their phosphate groups to form abasic oligonucleotides.

As used herein, an “allergen” refers to a substance that can induce an allergic or asthmatic response in a susceptible subject.

As used herein, an “allergic condition” refers to acquired hypersensitivity to a substance (allergen). Allergic conditions include eczema, allergic rhinitis or coryza, hay fever, allergic asthma, urticaria (hives), food allergies, and other atopic conditions.

As used herein, an “antigen” refers to a molecule capable of provoking a specific immune response. The term antigen broadly includes any type of molecule that is selectively bound by an antibody or by a T-cell antigen receptor and that is recognized by the immune system as foreign (i.e., danger) to the host. An antigen generally can initiate an adaptive immune response that includes generation of immunological memory for the antigen. An antigen can be a peptide or peptide fragment, or it can be any other type of molecule including a lipid, a nucleic acid, a polysaccharide, and any combination thereof. Antigens also specifically include allergens, self antigens, tumor antigens, alloantigens, and microbial antigens.

“Asthma” as used herein refers to a disorder of the respiratory system characterized by inflammation, narrowing of the airways and increased reactivity of the airways to inhaled agents. Asthma is frequently, although not exclusively, associated with an atopic or allergic condition.

An “autoimmune disease” as used herein refers to any of a number of clinically recognized organ-specific or systemic diseases involving an immune response directed against normal host cells or tissue. Autoimmune diseases are widely viewed as diseases caused by a breakdown of self-tolerance such that the adaptive immune system responds to self antigens and mediates cell and tissue damage. Non-limiting examples of autoimmune diseases include autoimmune type 1 (insulin-dependent) diabetes mellitus, multiple sclerosis, experimental allergic encephalomyelitis, ankylosing spondylitis, anti-glomerular basement membrane disease (e.g., Goodpasture's syndrome), atherosclerosis, autoimmune hepatitis, Behget's syndrome, Crohn's disease, Eaton-Lainbert myasthenic syndrome, glomerulonephritis, gluten-sensitive enteropathy, Graves' disease, Guillain-Barre syndrome, Hashimoto's thyroiditis, hemolytic anemias, idiopathic thrombocytopenic purpura, myasthenia gravis, pernicious anemia, primary biliary cirrhosis, psoriasis, Reiter's syndrome, rheumatic fever, rheumatoid arthritis, sclerosing cholangitis, Sjögren's syndrome, stiff-man syndrome, systemic lupus erythematosus, systemic sclerosis (scleroderma), Type I and Type II autoimmune polyglandular syndromes, uveitis, and Wegener's granulomatosis.

As used herein, a “C3 spacer” refers to a three-carbon, phosphate-containing unit having a structure provided as

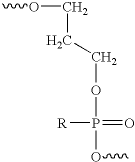

wherein R represents oxygen, sulfur, methyl, or O-alkyl.

As used herein, a “cancer” refers to a collection of cells of host origin having abnormal cell growth characterized by lack of regulation by external signals and by capacity to invade local or distant tissues which are normal. Cancers specifically include carcinomas, sarcomas, leukemias, and lymphomas. Cancers or tumors include but are not limited to biliary tract cancer; brain cancer; breast cancer; cervical cancer; choriocarcinoma; colon cancer; endometrial cancer; esophageal cancer; gastric cancer; glioma; intraepithelial neoplasms; lymphomas; liver cancer; lung cancer (e.g., small cell and non-small cell); melanoma; mesothelioma; neuroblastoma; oral cancer; ovarian cancer; pancreas cancer; prostate cancer; rectal cancer; renal cancer; retinoblastoma, sarcomas; skin cancer; testicular cancer; thyroid cancer; and other carcinomas and sarcomas.

As used herein, a “conjugate” refers to two or more entities bound to one another by any physicochemical means, including, but not limited to, covalent interaction, hydrophobic interaction, hydrogen bond interaction, or ionic interaction. The conjugate in one embodiment can include an abasic oligonucleotide and a therapeutic agent bound to one another directly. The conjugate in one embodiment can include an intermediate or linker entity between an abasic oligonucleotide and a therapeutic agent, such that the abasic oligonucleotide and the therapeutic agent are bound to one another indirectly. When the conjugate includes more than one abasic oligonucleotide or more than one therapeutic agent, then the various oligonucleotide and therapeutic agent components of the conjugate can be bound to one another directly, indirectly, or both directly and indirectly.

As used herein, a “CpG nucleic acid” refers to an immunostimulatory nucleic acid molecule, specifically including a CpG oligodeoxynucleotide (ODN) or, equivalently, a CpG oligonucleotide, that includes an unmethylated deoxycytidyl-deoxyguanosine (CpG) dinucleotide within a base sequence context termed a CpG motif. A CpG motif generally has the structure 5′-X₁X₂CGX₃X₄-3′, wherein C is unmethylated cytidine, G is guanosine, and X₁, X₂, X₃, and X₄ are nucleotides. In humans a preferred CpG motif has been reported to be 5′-GTCGTT-3′. In mice a preferred CpG motif has been reported to be 5′-GACGTT-3′. A CpG oligonucleotide in one embodiment is 6-100 nucleotides long. In one embodiment a CpG oligonucleotide is 6-40 nucleotides long. A CpG oligonucleotide in one embodiment is 6-24 nucleotides long. In one embodiment a CpG oligonucleotide is 6-20 nucleotides long.

Different classes of CpG ODN were recently characterized, all of which are included within the scope of the present invention. Vollmer J et al. (2004) Eur J Immunol. 34:251-62. The originally described B class is a very potent Th1 adjuvant, has anti-tumor activity, and stimulates strong B cell and natural killer (NK) cell activation or cytokine secretion. The A class have phosphorothioate G-rich 5′ and 3′ ends and a phosphodiester palindromic center, and they are especially potent in activating human plasmacytoid dendritic cells (pDC) to produce large amounts of interferon alpha (IFN-α). The recently described C class ODN is wholly phosphorothioate, has no poly-G stretches, has palindromic sequences combined with stimulatory CpG motifs, and strongly stimulate B cell and NK cell activation, as well as IFN-α production.

As used herein, an “effective amount” of a compound refers generally to an amount of that compound sufficient to achieve a desired biologic effect. Administration of an effective amount can involve administering a single dose or more than one dose. A pharmaceutically effective amount for any particular application can vary depending on such factors as the disease or condition being treated, the particular compound or treatment being administered, the size of the subject, or the severity of the disease or condition. One of ordinary skill in the art can empirically determine the effective amount of a particular conjugate of the invention without necessitating undue experimentation.

An “immunostimulatory nucleic acid molecule” refers to a nucleic acid molecule which stimulates (e.g., has a mitogenic effect on, or induces or increases cytokine expression by) a vertebrate leukocyte. In one embodiment an immunostimulatory nucleic acid is a DNA molecule. In one embodiment an immunostimulatory nucleic acid is a CpG oligonucleotide. An immunostimulatory nucleic acid molecule can be double-stranded or single-stranded. Immunostimulatory nucleic acid molecules specifically include, but are not limited to, immunostimulatory oligonucleotides such as are disclosed in U.S. Pat. Nos. 6,194,388, 6,207,646, 6,214,806, 6,218,371, 6,239,116, 6,339,068, 6,429,199, and 6,653,292. In one embodiment an immunostimulatory nucleic acid is an RNA molecule. Immunostimulatory nucleic acid molecules further specifically include, but are not limited to, immunostimulatory RNA oligonucleotides such as are disclosed in published international patent application WO 03/086280.

An “infection” refers to an abnormal collection of infectious microorganisms or infectious agents present in a host subject. Infectious microorganisms and infectious agents include viruses, bacteria, fungi, and parasites.

An “inflammatory response” refers to any antigen-nonspecific immune response in which there is local accumulation of activated leukocytes at a site of infection, toxin exposure, or cell injury.

A “linker” refers to a chemical moiety which connects one chemical moiety to another chemical moiety. A linker can be chemically similar to or chemically distinct from a chemical moiety to which it is connected. Linkers will typically, but not necessarily, be covalently coupled to the chemical moieties it connects.

The term “pharmaceutically acceptable carrier” as used herein means one or more compatible solid or liquid filler, diluent, or encapsulating substances which are suitable for administration into a subject. The term “carrier” denotes an organic or inorganic ingredient, natural or synthetic, with which the active ingredient is combined to facilitate the application.

A “small molecule” as used herein refers to an organic or inorganic molecule, either natural (i.e., found in nature) or non-natural (i.e., not found in nature), which has a molecular weight of less than about 1.5 kilodaltons (kDa). Most pharmaceutical agents (i.e., drugs), except for certain macromolecular biologicals, are small molecules.

A “subject” shall mean a human or vertebrate animal including a dog, cat, horse, cow, pig, sheep, goat, chicken, monkey, rat, mouse, etc.

As used herein, a “therapeutic agent” refers to any composition useful in the treatment or diagnosis of a disease or condition of a subject. In one embodiment the therapeutic agent is an antigen. In one embodiment the therapeutic agent is a nucleic acid molecule other than the abasic oligonucleotide. In one embodiment the therapeutic agent is an immunostimulatory nucleic acid molecule. In one embodiment the therapeutic agent is an immunoinhibitory nucleic acid (also known as an inhibitory nucleic acid). In one embodiment the therapeutic agent is a small molecule. In various embodiments the therapeutic agent can belong to any of a number of well known classes of drugs, including, without limitation, antibiotics, anti-inflammatory agents, hormones, antihistamines, reverse transcriptase inhibitors, antimetabolites, antineoplastics, antiarrhythmics, prostaglandins, nucleoside analogues, oligonucleotides, and radionuclides.

A “Toll-like receptor” (and equivalently “TLR”) refers generally to any of a family of highly conserved pattern recognition receptors that are involved in innate immunity. Unless otherwise specified, the term TLR as used herein shall refer to a TLR polypeptide. TLRs currently include ten members (TLR1-TLR10) characterized by structural features which include an extracellular domain with leucine-rich repeats and an intracellular Toll-like domain that is involved in immune activation signaling. Akira S (2001) Adv Immunol 78:1-56; Medzhitov R et al. (2000) Immunol Rev 173:89-97. Nucleotide and amino acid sequences for various TLRs are publicly available from GenBank and other public databases.

A “TLR signaling agonist” is any compound that specifically induces or increases intracellular signaling involving a TLR. The induction or increase in signaling can be direct or indirect, acting at the level of a TLR interacting with its ligand or intracellular adaptor molecule (e.g., MyD88) or at the level of downstream signaling. TLR agonists are typically specific to a particular TLR, although there can be some overlap among different TLRs. A TLR signaling agonist can include a natural ligand for the TLR (i.e., a ligand found in nature that binds the TLR). A TLR signaling agonist can include a non-natural ligand for the TLR (i.e., a ligand not found in nature that binds the TLR). In one embodiment a TLR9 signaling agonist is a CpG nucleic acid. A TLR signaling agonist is in one embodiment a small molecule.

A “TLR signaling antagonist” refers to any compound that specifically interferes with or reduces intracellular signaling involving a TLR. The interference can be direct or indirect, acting at the level of a TLR interacting with its ligand or intracellular adaptor molecule (e.g., MyD88) or at the level of downstream signaling. TLR antagonists are typically specific to a particular TLR, although there can be some overlap among different TLRs. A TLR signaling antagonist can include a competitor of a natural ligand for the TLR. In one embodiment a TLR signaling antagonist is an inhibitory oligonucleotide. See, for example, Stunz L L et al. (2002) Eur J Immunol. 32:1212-22; Lenert P et al. (2003) Antisense Nucleic Acid Drug Dev. 13:143-50. A TLR signaling antagonist is in one embodiment a small molecule. See, for example, U.S. Pat. Nos. 6,221,882, 6,399,630, 6,479,504, 6,521,637, and U.S. Published Patent Appls. 2003-0232856 A1 and 2005-0119273 A1.

As used herein, “TLR3” refers to Toll-like receptor 3. Human TLR3 is a 904 amino acid protein expressed by dendritic cells. Muzio M et al. (2000) J Immunol 164:5998-6004. It was recently reported that ligands of TLR3 include polyinosine-polycytidylic acid (poly(I:C)) and double-stranded RNA (dsRNA). By stimulating kidney cells expressing one of a range of TLRs with poly(I:C), Alexopoulou et al. reported that only cells expressing TLR3 respond by activating NF-κB. Alexopoulou L et al. (2001) Nature 413:732-8. Alexopoulou et al. also reported that wildtype cells stimulated with poly(I:C) activate NF-κB and produce inflammatory cytokines IL-6, IL-12, and TNF-α, whereas the corresponding responses of TLR3^{-/-} cells were significantly impaired. In contrast, TLR3^{-/-} cells responded equivalently to wildtype cells in response to lipopolysaccharide, peptidoglycan, and CpG dinucleotides. Analysis of MyD88^{-/-} cells indicated that this adaptor protein is involved in dsRNA-induced production of cytokines and proliferative responses, although activation of NF-κB and MAP kinases are not affected, indicating distinct pathways for these cellular responses. Alexopoulou et al. proposed that TLR3 may have a role in host defense against viruses.

As used herein, “TLR7” refers to Toll-like receptor 7. Nucleotide and amino acid sequences of human and murine TLR7 are known. See, for example, GenBank Accession Nos. AF240467, AF245702, NM_{—}016562, AF334942, NM_{—}133211; and AAF60188, AAF78035, NP_{—}057646, AAL73191, AAL73192. Human TLR7 is reported to be 1049 amino acids long. Murine TLR7 is reported to be 1050 amino acids long. TLR7 polypeptide includes an extracellular domain having leucine-rich repeat region, a transmembrane domain, and an intracellular domain that includes a Toll/IL-1 receptor (TIR) domain.

As used herein, “TLR8” refers to Toll-like receptor 8. Nucleotide and amino acid sequences of human and murine TLR8 are known. See, for example, GenBank Accession Nos. AF246971, AF245703, NM 016610, XM_{—}045706, AY035890, NM_{—}133212; and AAF64061, AAF78036, NP_{—}057694, XP_{—}045706, AAK62677, NP_{—}573475. Human TLR8 is reported to exist in at least two isoforms, one 1041 amino acids long and the other 1059 amino acids long. The shorter of these two isoforms is believed to be more important. Murine TLR8 is 1032 amino acids long. TLR8 polypeptide includes an extracellular domain having leucine-rich repeat region, a transmembrane domain, and an intracellular domain that includes a TIR domain.

As used herein, “TLR9” refers to Toll-like receptor 9. Nucleotide and amino acid sequences of human and murine TLR9 are known. See, for example, GenBank Accession Nos. NM_{—}017442, AF259262, AB045180, AF245704, AB045181, AF348140, AF314224, NM_{—}031178; and NP_{—}059138, AAF 72189, BAB19259, AAF78037, BAB19260, AAK29625, AAK28488, NP_{—}112455. Human TLR9 is reported to exist in at least two isoforms, one 1032 amino acids long and the other 1055 amino acids long. The shorter of these two isoforms is believed to be more important. Murine TLR9 is 1032 amino acids long. TLR9 polypeptide includes an extracellular domain having leucine-rich repeat region, a transmembrane domain, and an intracellular domain that includes a TIR domain.

The term “treat” as used herein refers to preventing, slowing, reducing progression of, halting, or eliminating a measurable sign or symptom of a disease or disorder of a subject.

A “unit” as used herein in reference to an oligonucleotide or polymer refers to a chemical entity that is a structural unit (sometimes referred to as a monomer unit) of the oligonucleotide or polymer. For example, in one embodiment a unit as used herein can refer to an abasic deoxyribonucleotide. As another example, in one embodiment a unit refers to an abasic ribonucleotide. As yet another example, in one embodiment a unit refers to a C3 spacer as described above. In one embodiment each unit is identical to every other unit, in which case the unit can also be referred to as a repeat unit and the oligonucleotide or polymer is a homopolymer. In one embodiment at least one unit is nonidentical to at least one other unit, in which case the oligonucleotide or polymer is a copolymer.

In one aspect the invention provides a composition that is a conjugate including an abasic oligonucleotide 10-40 units long and a therapeutic agent. As described above, an abasic oligonucleotide resembles a backbone of a DNA or an RNA molecule, wherein the nucleobases (e.g., adenine, cytosine, thymine, uracil, and guanine) and optionally the sugar residues are absent. In one embodiment the β-ribose unit or β-D-2′-deoxyribose unit is replaced by a three-carbon unit corresponding to a C3 spacer derived from propane-1,3-diol. Alternatively, a β-ribose unit or a β-D-2′-deoxyribose unit can be replaced by a modified sugar unit, wherein the modified sugar unit is for example selected from β-D-ribose, α-D-2′-deoxyribose, L-2′-deoxyribose, 2′-F-2′-deoxyribose, 2′-O—(C₁-C₆)alkyl-ribose, 2′-O-methylribose, 2′-O—(C₂-C₆)alkenyl-ribose, 2′-[O—(C₁-C₆)alkyl-O—(C₁-C₆)alkyl]-ribose, 2′—NH₂-2′-deoxyribose, β-D-xylo-furanose, α-arabinofuranose, 2,4-dideoxy-β-D-erythro-hexo-pyranose, and carbocyclic (described, for example, in Froehler J (1992) Am Chem Soc 114:8320) and/or open-chain sugar analogs (described, for example, in Vandendriessche et al. (1993) Tetrahedron 49:7223) and/or bicyclosugar analogs (described, for example, in Tarkov M et al. (1993) Helv Chim Acta 76:481).

The abasic oligonucleotide is a polymer of identical or non-identical units connected to each other by phosphate-containing linkages. In one embodiment the phosphate-containing linkages are all stabilized, i.e., relatively resistant to in vivo degradation, e.g., via an exonuclease or endonuclease. Such stabilized phosphate-containing linkages can include, without limitation, phosphorothioate, phosphorodithioate, methylphosphonate, methylphosphorothiate. In another embodiment, all the phosphate-containing linkages are phosphodiester and are relatively susceptible to in vivo degradation, e.g., via an exonuclease or endonuclease. In yet another embodiment at least one of the phosphate-containing linkages is phosphodiester while other phosphate-containing linkages are stabilized. In one embodiment at least one of the phosphate-containing linkages is a phosphodiester linkage and at least one of the phosphate-containing linkages is a phosphorothioate linkage. The inclusion of phosphodiester linkages and the position of phosphodiester linkages can affect the pharmacokinetics of the conjugate, for example by providing sites of greater susceptibility to nuclease cleavage, resulting in release of the therapeutic agent, decrease in size of the abasic oligonucleotide below a length that is efficiently taken up by cells, or both.

In embodiments in which there is a mixture of two types of phosphate-containing linkages, e.g., phosphodiester linkages and phosphorothioate linkages, the ratio of one type of linkage to another can range from 1:(n−2) to (n−2): 1 for any abasic oligonucleotide that is n units long (i.e., having n−1 inter-unit linkages). In other embodiments there is a mixture of at least three types of phosphate-containing linkages, some or all of which can be stabilized.

In one embodiment the abasic oligonucleotide is a homopolymer of abasic deoxyribonucleotides. In another embodiment the abasic oligonucleotide is a homopolymer of abasic ribonucleotides. In another embodiment the abasic oligonucleotide is a homopolymer of C3 spacers. In each of the foregoing homopolymers the phosphate-containing linkages connecting adjacent units can be homogeneous or they can be heterogeneous. In one embodiment the phosphate-containing linkages connecting adjacent units are heterogeneous and include at least one phosphodiester linkage and at least one stabilized linkage. In one embodiment the phosphate-containing linkages connecting adjacent units include at least one phosphodiester linkage and at least one phosphorothioate linkage. In one embodiment each and every phosphate-containing linkage is stabilized. In one embodiment each and every phosphate-containing linkage is phosphorothioate.

In another embodiment the abasic oligonucleotide is a heteropolymer of abasic ribonucleotides and abasic deoxyribonucleotides. The abasic ribonucleotides and abasic deoxyribonucleotides in this embodiment can be present in any integer ratio that is consistent with the overall number of units in the abasic oligonucleotide. The ratio of one type of unit to another can thus range from 1: (n−1) to (n−1): 1 for any abasic oligonucleotide that is n units long. In addition, the phosphate-containing linkages connecting adjacent units in this embodiment can be either homogeneous, e.g., all phosphorothioate, or they can be heterogeneous, e.g., at least one phosphodiester linkage and at least one stabilized linkage. In one embodiment each and every phosphate-containing linkage is stabilized. In one embodiment each and every phosphate-containing linkage is phosphorothioate.

In yet other embodiments the abasic oligonucleotide is a heteropolymer of any combination of abasic ribonucleotides, abasic deoxyribonucleotides, and C3 spacers derived from propane-1,3-diol. Such heteropolymers can have homogeneous or heterogeneous phosphate-containing linkages interconnecting various adjacent units. In one embodiment each and every phosphate-containing linkage is stabilized. In one embodiment each and every phosphate-containing linkage is phosphorothioate.

For use in the instant invention, the abasic oligonucleotides of the invention can be synthesized de novo using any of a number of procedures well known in the art. For example, such methods include the β-cyanoethyl phosphoramidite method (Beaucage S L et al. (1981) Tetrahedron Lett 22:1859) and the nucleoside H-phosphonate method (Garegg et al. (1986) Tetrahedron Lett 27:4051-4; Froehler et al. (1986) Nucl Acid Res 14:5399-407; Garegg et al. (1986) Tetrahedron Lett 27:4055-8; Gaffney et al. (1988) Tetrahedron Lett 29:2619-22). These chemistries can be performed by a variety of automated nucleic acid synthesizers available in the market.

Abasic oligonucleotides incorporating modified backbones such as phosphorothioates can be synthesized using automated techniques employing either phosphoramidate or H-phosphonate chemistries. Aryl- and alkyl-phosphonates can be made, e.g., as described in U.S. Pat. No. 4,469,863; and alkylphosphotriesters (in which the charged oxygen moiety is alkylated as described in U.S. Pat. No. 5,023,243 and European Pat. No. 092,574) can be prepared by automated solid phase synthesis using commercially available reagents. Methods for making other backbone modifications and substitutions have been described. See, for example, Uhlmann E et al. (1990) Chem Rev 90:544 and Goodchild J (1990) Bioconjugate Chem 1:165.

The conjugates of the invention include an abasic oligonucleotide, as described above, linked to a therapeutic agent. According to this and other aspects of the invention, in one embodiment the therapeutic agent is an antigen. The antigen in various embodiments can be an antigen characteristic of an infectious agent, a cancer antigen, an allergen, an antigen characteristic of a cell or tissue transplant (e.g., an alloantigen), or an antigen characteristic of an autoimmune disease.

In one embodiment the therapeutic agent is an antigen characteristic of an infectious agent. The term “antigen characteristic of an infectious agent” refers to an antigen expressed by or derived from an infectious microorganism or other infectious agent. Infectious microorgansims and other infectious agents include bacteria, viruses, fungi, and parasites.

Infectious bacteria include, but are not limited to, gram negative and gram positive bacteria. Gram positive bacteria include, but are not limited to Pasteurella species, Staphylococci species, and Streptococcus species. Gram negative bacteria include, but are not limited to, Escherichia coli, Pseudomonas species, and Salmonella species. Specific examples of infectious bacteria include but are not limited to: Helicobacter pyloris, Borrelia burgdorferi, Legionella pneumophilia, Mycobacteria sps (e.g., M. tuberculosis, M. avium, M. intracellulare, M. kansasii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic species), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasturella multocida, Bacteroides sp., Fusobacterium nucleatum, Streptobacillus moniliforinis, Treponema pallidum, Treponema pertenue, Leptospira, Rickettsia, and Actinomyces israelli.

Viruses are small infectious agents which generally contain a nucleic acid core and a protein coat, but are not independently living organisms. Viruses can also take the form of infectious nucleic acids lacking a protein. A virus cannot survive in the absence of a living cell within which it can replicate. Viruses enter specific living cells either by endocytosis or direct injection of DNA (phage) and multiply, causing disease. The multiplied virus can then be released and infect additional cells. Some viruses are DNA-containing viruses and others are RNA-containing viruses.

Viruses include, but are not limited to, enteroviruses (including, but not limited to, viruses that belong to the family Picornaviridae, such as polio virus, coxsackie virus, echo virus), rotaviruses, adenovirus, hepatitis virus. Specific examples of viruses that have been found in humans include but are not limited to: Retroviridae (e.g., human immunodeficiency viruses, such as HIV-1 (also referred to as HTLV-III, LAV or HTLV-III/LAV, or HIV-III; and other isolates, such as HIV-LP; Picornaviridae (e.g., polio viruses, hepatitis A virus; enteroviruses, human Coxsackie viruses, rhinoviruses, echoviruses); Calciviridae (e.g., strains that cause gastroenteritis); Togaviridae (e.g., equine encephalitis viruses, rubella viruses); Flaviviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Pararnyxoviridae (e.g., parainfluenza viruses, mumps virus, measles virus, respiratory syncytial virus); Orthomyxoviridae (e.g., influenza viruses); Bunyaviridae (e.g., Hantaan viruses, bunya viruses, phleboviruses and Nairo viruses); Arenaviridae (hemorrhagic fever viruses); Reoviridae (e.g., reoviruses, orbiviurses and rotaviruses); Birnaviridae; Hepadnaviridae (Hepatitis B virus); Parvoviridae (parvoviruses); Papovaviridae (papillomaviruses, polyoma viruses); Adenoviridae (most adenoviruses); Herpesviridae (herpes simplex virus (HSV) 1 and 2, varicella zoster virus, cytomegalovirus (CMV)); Poxyiridae (variola viruses, vaccinia viruses, pox viruses); Iridoviridae (e.g., African swine fever virus); and unclassified viruses (e.g., the agent of delta hepatitis (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses).

Fungi are eukaryotic organisms, only a few of which cause infection in vertebrate mammals. Because fungi are eukaryotic organisms, they differ significantly from prokaryotic bacteria in size, structural organization, life cycle and mechanism of multiplication. Fungi are classified generally based on morphological features, modes of reproduction and culture characteristics. Although fungi can cause different types of disease in subjects, such as respiratory allergies following inhalation of fungal antigens, fungal intoxication due to ingestion of toxic substances, such as Amanita phalloides toxin and phallotoxin produced by poisonous mushrooms and aflatoxins, produced by Aspergillus species, not all fungi cause infectious disease.

Infectious fungi can cause systemic or superficial infections. Primary systemic infection can occur in normal healthy subjects, and opportunistic infections are most frequently found in immunocompromised subjects. The most common fungal agents causing primary systemic infection include Blastomyces, Coccidioides, and Histoplasma. Common fungi causing opportunistic infection in immunocompromised or immunosuppressed subjects include, but are not limited to, Candida albicans, Cryptococcus neoformans, and various Aspergillus species. Systemic fungal infections are invasive infections of the internal organs. The organism usually enters the body through the lungs, gastrointestinal tract, or intravenous catheters. These types of infections can be caused by primary pathogenic fungi or opportunistic fungi.

Superficial fungal infections involve growth of fungi on an external surface without invasion of internal tissues. Typical superficial fungal infections include cutaneous fungal infections involving skin, hair, or nails.

Diseases associated with fungal infection include aspergillosis, blastomycosis, candidiasis, chromoblastomycosis, coccidioidomycosis, cryptococcosis, fungal eye infections, fungal hair, nail, and skin infections, histoplasmosis, lobomycosis, mycetoma, otomycosis, paracoccidioidomycosis, disseminated Penicillium marneffei, phaeohyphomycosis, rhinosporidioisis, sporotrichosis, and zygomycosis.

Parasites are organisms which depend upon other organisms in order to survive and thus must enter, or infect, another organism to continue their life cycle. The infected organism, i.e., the host, provides both nutrition and habitat to the parasite. Although in its broadest sense the term parasite can include all infectious agents (i.e., bacteria, viruses, fungi, protozoa and helminths), generally speaking, the term is used to refer solely to protozoa, helminths, and ectoparasitic arthropods (e.g., ticks, mites, etc.). Protozoa are single-celled organisms which can replicate both intracellularly and extracellularly, particularly in the blood, intestinal tract or the extracellular matrix of tissues. Helminths are multicellular organisms which almost always are extracellular (an exception being Trichinella spp.). Helminths normally require exit from a primary host and transmission into a secondary host in order to replicate. In contrast to these aforementioned classes, ectoparasitic arthropods form a parasitic relationship with the external surface of the host body.

Parasites include intracellular parasites and obligate intracellular parasites. Examples of parasites include but are not limited to Plasmodium falciparum, Plasmodium ovale, Plasmodium malariae, Plasmdodium vivax, Plasmodium knowlesi, Babesia microti, Babesia divergens, Trypanosoma cruzi, Toxoplasma gondii, Trichinella spiralis, Leishmania major, Leishmania donovani, Leishmania braziliensis, Leishmania tropica, Trypanosoma gambiense, Trypanosoma rhodesiense and Schistosoma mansoni.

Other medically relevant microorganisms have been described extensively in the literature, e.g., see C. G. A Thomas, Medical Microbiology, Bailliere Tindall, Great Britain 1983, the entire contents of which is hereby incorporated by reference. Each of the foregoing lists is illustrative and is not intended to be limiting.

In one embodiment the therapeutic agent is a cancer antigen. The terms “cancer antigen” and “tumor antigen” are used interchangeably herein to refer to antigens which are differentially expressed by cancer cells and can thereby be exploited in order to target cancer cells. Cancer antigens are antigens which can potentially stimulate apparently tumor-specific immune responses. Some of these antigens are encoded, although not necessarily expressed, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as those carried on RNA and DNA tumor viruses. Examples of tumor antigens include MAGE, MART-1/Melan-A, gp100, Dipeptidyl peptidase IV (DPPIV), adenosine deaminase-binding protein (ADAbp), cyclophilin b, Colorectal associated antigen (CRC)—C017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100^{Pmel117}, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, imp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, and c-erbB-2.

Cancers or tumors and tumor antigens associated with such tumors (but not exclusively), include acute lymphoblastic leukemia (etv6; aml1; cyclophilin b), B cell lymphoma (Ig-idiotype), glioma (E-cadherin; α-catenin; β-catenin; γ-catenin; p120ctn), bladder cancer (p221 ras), biliary cancer (p221 ras), breast cancer (MUC family; HER2/neu; c-erbB-2), cervical carcinoma (p53; p21ras), colon carcinoma (p21ras; HER2/neu; c-erbB-2; MUC family), colorectal cancer (Colorectal associated antigen (CRC)—C017-1A/GA733; APC), choriocarcinoma (CEA), epithelial cell cancer (cyclophilin b), gastric cancer (HER2/neu; c-erbB-2; ga733 glycoprotein), hepatocellular cancer (α-fetoprotein), Hodgkins lymphoma (Imp-1; EBNA-1), lung cancer (CEA; MAGE-3; NY-ESO-1), lymphoid cell-derived leukemia (cyclophilin b), melanoma (p115 protein, gp75, oncofetal antigen, GM2 and GD2 gangliosides), myeloma (MUC family; p21ras), non-small cell lung carcinoma (HER2/neu; c-erbB-2), nasopharyngeal cancer (Imp-1; EBNA-1), ovarian cancer (MUC family; HER2/neu; c-erbB-2), prostate cancer (Prostate Specific Antigen (PSA) and its immunogenic epitopes PSA-1, PSA-2, and PSA-3; PSMA; HER2/neu; c-erbB-2), pancreatic cancer (p21ras; MUC family; HER2/neu; c-erbB-2; ga733 glycoprotein), renal cancer (HER2/neu; c-erbB-2), squamous cell cancers of cervix and esophagus (viral products such as human papilloma virus proteins), testicular cancer (NY-ESO-1), T-cell leukemia (HTLV-1 epitopes), and melanoma (Melan-A/MART-1; cdc27; MAGE-3; p21ras; gp100^{PmeI117}).

For examples of tumor antigens which bind to either or both MHC class I and MHC class II molecules, see the following references: Aarnoudse et al. Int J Cancer 82:442-448, 1999; Boel et al. Immunity 2:167-175, 1995; Brandle et al. J Exp Med 183:2501-2508, 1996; Brichard et al. Eur J Immunol 26:224-230, 1996; Brossart et al. Cancer Res 58:732-736, 1998; Castelli et al. J Exp Med 181:363-368, 1995; Castelli et al. J Immunol 162:1739-1748, 1999; Chaux et al. J Exp Med 189:767-778, 1999; Chaux et al. J Immunol 163:2928-2936, 1999; Chiari et al. Cancer Res 59:5785-5792, 1999; Correale et al. J Natl Cancer Inst 89:293-300, 1997; Coulie et al. Proc Natl Acad Sci USA 92:7976-7980, 1995; Coulie, Stem Cells 13:393-403, 1995; Cox et al. Science 264:716-719, 1994; De Backer et al. Cancer Res 59:3157-3165, 1999; Duffour et al. Eur J Immunol 29:3329-3337, 1999; Fisk et al. J Exp Med 181:2109-2117, 1995; Fujie et al. Int J Cancer 80:169-172, 1999; Gaudin et al. J Immunol 162:1730-1738, 1999; Gaugler et al. J Exp Med 179:921-930, 1994; Gjertsen et al. Int J Cancer 72:784-790, 1997; Gueguen et al. J Immunol 160:6188-6194, 1998; Guilloux et al. J Exp Med 183:1173-1183, 1996; Herman et al. Immunogenetics 43:377-383, 1996; Hogan et al. Cancer Res 58:5144-5150, 1998; Huang et al. J Immunol 162:6849-6854, 1999; Ikeda et al. Immunity 6:199-208, 1997; Jager et al. J Exp Med 187:265-270, 1998; Kang et al. J Immunol 155:1343-1348, 1995; Kawakami et al. J Exp Med 180:347-352, 1994; Kawakami et al. J Immunol 154:3961-3968, 1995; Kawakami et al. J Immunol 161:6985-6992, 1998; Kawakami et al. Proc Natl Acad Sci USA 91:6458-6462, 1994; Kawashima et al. Hum Immunol 59:1-14, 1998; Kittlesen et al. J Immunol 160:2099-2106, 1998; Kobayashi et al. Cancer Research 58:296-301, 1998; Lupetti et al. J Exp Med 188:1005-1016, 1998; Mandruzzato et al. J Exp Med 186:785-793, 1997; Manici et al. J Exp Med 189:871-876, 1999; Morel et al. Int J Cancer 83:755-759, 1999; Oiso et al. Int J Cancer 81:387-394, 1999; Parkhurst et al. Cancer Research 58:4895-4901, 1998; Pieper et al. J Exp Med 189:757-765, 1999; Robbins et al. J Exp Med 183:1185-1192, 1996; Robbins et al. J Immunol 159:303-308, 1997; Ronsin et al. J Immunol 163:483-490, 1999; Röpke et al. Proc Natl Acad Sci USA 93:14704-14707, 1996; Schneider et al. Int J Cancer 75:451-458, 1998; Skipper et al. J Exp Med 183:527-534, 1996; Skipper et al. J Immunol 157:5027-5033, 1996; Tahara et al. Clin Cancer Res 5:2236-2241, 1999; Tanaka et al. Cancer Res 57:4465-4468, 1997; Tanzarella et al. Cancer Res 59:2668-2674, 1999; ten Bosch et al. Blood 88:3522-3527, 1996; Topalian et al. J Exp Med 183:1965-1971, 1996; Traversari et al. J Exp Med 176:1453-1457, 1992; Tsai et al. J Immunol 158:1796-1802, 1997; Tsang et al. J Natl Cancer Inst 87:982-990, 1995; Van den Eynde et al. J Exp Med 182:689-698, 1995; van der Bruggen et al. Eur J Immunol 24:2134-2140, 1994; van der Bruggen et al. Eur J Immunol 24:3038-3043, 1994; Vonderheide et al. Immunity 10:673-679, 1999; Wang et al. J Exp Med 183:1131-1140, 1996; Wang et al. J Exp Med 184:2207-2216, 1996; Wanget al. J Immunol 161:3596-3606, 1998; Wang et al. Science 284:1351-1354, 1999; Wolfel et al. Eur J Immunol 24:759-764, 1994; Wölfel et al. Science 269:1281-1284, 1995; Zorn et al. Eur J Immunol 29:602-607, 1999. These antigens as well as others are disclosed in published international patent application WO 99/14326.

In one embodiment the therapeutic agent that is an antigen is an allergen. As mentioned above, an allergen is a substance that can induce an allergic or asthmatic response in a susceptible subject. Allergens generally trigger an allergic response which is mediated by IgE antibody. The method and preparations of this invention extend to a broad class of such allergens and fragments of allergens or haptens acting as allergens.

The list of allergens is enormous and can include pollens, insect venoms, animal dander, dust, fungal spores and drugs (e.g., penicillin). Examples of natural, animal and plant allergens include proteins specific to the following genera: Agropyron (e.g., Agropyron repens); Agrostis (e.g., Agrostis alba); Alder; Alnus (Alnus gultinosa); Alternaria (Alternaria alternata); Ambrosia (Ambrosia artemiisfolia; Anthoxanthum (e.g., Anthoxanthum odoratum); Apis (e.g., Apis multiflorum); Arrhenatherum (e.g., Arrhenatherum elatius); Artemisia (Artemisia vulgaris); Avena (e.g., Avena sativa); Betula (Betula verrucosa); Blattella (e.g., Blattella germanica); Bromus (e.g., Bromus inermis); Canine (Canis familiaris); Chamaecyparis (e.g., Chamaecyparis obtusa); Cryptomeria (Cryptomeria japonica); Cupressus (e.g., Cupressus sempervirens, Cupressus arizonica and Cupressus macrocarpa); Dactylis (e.g., Dactylis gloinerata); Dermatophagoides (e.g., Dermatophagoides farinae); Felis (Felis domesticus); Festuca (e.g., Festuca elatior); Holcus (e.g., Holcus lanatus); Juniperus (e.g., Juniperus sabinoides, Juniperus virginiana, Juniperus communis and Juniperus ashei); Lolium (e.g., Lolium perenne or Lolium multiflorum); Olea (Olea europa); Parietaria (e.g., Parietaria officinalis or Parietaria judaica); Paspalum (e.g., Paspalum notatum); Periplaneta (e.g., Periplaneta americana); Phalaris (e.g., Phalaris arundinacea); Phleum (e.g., Phleum pratense); Plantago (e.g., Plantago lanceolata); Poa (e.g., Poapratensis or Poa compressa); Quercus (Quercus alba); Secale (e.g., Secale cereale); Sorghum (e.g., Sorghum halepensis); Thuya (e.g., Thuya orientalis); and Triticum (e.g., Triticum aestivum).

An allergic reaction occurs when tissue-sensitizing immunoglobulin of the IgE type reacts with foreign allergen. The IgE antibody is bound to mast cells and/or basophils, and these specialized cells release chemical mediators (vasoactive amines) of the allergic reaction when stimulated to do so by allergens bridging the ends of the antibody molecule. Histamine, platelet activating factor, arachidonic acid metabolites, and serotonin are among the best known mediators of allergic reactions in man. Histamine and the other vasoactive amines are normally stored in mast cells and basophil leukocytes. The mast cells are dispersed throughout animal tissue and the basophils circulate within the vascular system. These cells manufacture and store histamine within the cell unless the specialized sequence of events involving IgE binding occurs to trigger its release.

The symptoms of the allergic reaction vary, depending on the location within the body where the IgE reacts with the antigen. If the reaction occurs along the respiratory epithelium, the symptoms are sneezing, coughing and asthmatic reactions. If the interaction occurs in the digestive tract, as in the case of food allergies, abdominal pain and diarrhea are common. Systemic reactions, for example following a bee sting, can be severe and often life-threatening.

Delayed-type hypersensitivity, also known as type IV allergy reaction, is an allergic reaction characterized by a delay period of at least 12 hours from invasion of the antigen into the allergic subject until appearance of the inflammatory or immune reaction. The T lymphocytes (sensitized T lymphocytes) of individuals in an allergic condition react with the antigen, triggering the T lymphocytes to release lymphokines (macrophage migration inhibitory factor (MIF), macrophage activating factor (MAF), mitogenic factor (MF), skin-reactive factor (SRF), chemotactic factor, neovascularization-accelerating factor, etc.), which function as inflammation mediators, and the biological activity of these lymphokines, together with the direct and indirect effects of locally appearing lymphocytes and other inflammatory immune cells, give rise to the type IV allergy reaction. Delayed allergy reactions include tuberculin type reaction, homograft rejection reaction, cell-dependent type protective reaction, contact dermatitis hypersensitivity reaction, and the like, which are known to be most strongly suppressed by steroidal agents. Consequently, steroidal agents are effective against diseases which are caused by delayed allergy reactions. Long-term use of steroidal agents at concentrations currently being used can, however, lead to the serious side-effect known as steroid dependence. The methods of the invention solve some of these problems, by providing for lower and fewer doses to be administered.

Immediate hypersensitivity (or anaphylactic response) is a form of allergic reaction which develops very quickly, i.e., within seconds or minutes of exposure of the patient to the causative allergen, and it is mediated by IgE antibodies made by B lymphocytes. In nonallergic patients, there is no IgE antibody of clinical relevance; but, in a person suffering with allergic diseases, IgE antibody mediates immediate hypersensitivity by sensitizing mast cells which are abundant in the skin, lymphoid organs, in the membranes of the eye, nose and mouth, and in the respiratory tract and intestines.

Mast cells have surface receptors for IgE, and the IgE antibodies in allergy-suffering patients become bound to them. As discussed briefly above, when the bound IgE is subsequently contacted by the appropriate allergen, the mast cell is caused to degranulate and to release various substances called bioactive mediators, such as histamine, into the surrounding tissue. It is the biologic activity of these substances which is responsible for the clinical symptoms typical of immediate hypersensitivity; namely, contraction of smooth muscle in the airways or the intestine, the dilation of small blood vessels and the increase in their permeability to water and plasma proteins, the secretion of thick sticky mucus, and in the skin, redness, swelling and the stimulation of nerve endings that results in itching or pain.

Symptoms of asthma include recurrent episodes of wheezing, breathlessness, and chest tightness, and coughing, resulting from airflow obstruction. Airway inflammation associated with asthma can be detected through observation of a number of physiological changes, such as, denudation of airway epithelium, collagen deposition beneath basement membrane, edema, mast cell activation, inflammatory cell infiltration, including neutrophils, eosinophils, and lymphocytes. As a result of the airway inflammation, asthma patients often experience airway hyper-responsiveness, airflow limitation, respiratory symptoms, and disease chronicity. Airflow limitations include acute bronchoconstriction, airway edema, mucous plug formation, and airway remodeling, features which often lead to bronchial obstruction. In some cases of asthma, sub-basement membrane fibrosis may occur, leading to persistent abnormalities in lung function.

Research over the past several years has revealed that asthma likely results from complex interactions among inflammatory cells, mediators, and other cells and tissues resident in the airway. Mast cells, eosinophils, epithelial cells, macrophages, and activated T-cells all play an important role in the inflammatory process associated with asthma. Djukanovic R et al. (1990) Am Rev Respir Dis 142:434-7. It is believed that these cells can influence airway function through secretion of preformed and newly synthesized mediators which can act directly or indirectly on the local tissue. It has also been recognized that subpopulations of T lymphocytes (Th2) play an important role in regulating allergic inflammation in the airway by releasing selective cytokines and establishing disease chronicity. Robinson D S et al. (1992) N Engl J Med 326:298-304.

Asthma is a complex disorder which arises at different stages in development and can be classified based on the degree of symptoms as acute, subacute or chronic. An acute inflammatory response is associated with an early recruitment of cells into the airway. The subacute inflammatory response involves the recruitment of cells as well as the activation of resident cells causing a more persistent pattern of inflammation. Chronic inflammatory response is characterized by a persistent level of cell damage and an ongoing repair process, which may result in permanent abnormalities in the airway.

In one embodiment the therapeutic agent is an antigen that is characteristic of an autoimmune disease. As mentioned above, autoimmune diseases are believed to reflect loss or breakdown of normal mechanisms of self-tolerance, i.e., tolerance to self antigens. While autoimmune diseases may arise against a single self antigen, in many cases autoimmune diseases evolve, through a process known as epitope spreading, to include immune reactivity toward a number of self antigens. While the list of self antigens possibly involved in autoimmune disease is potentially enormous, certain antigens characteristic of an autoimmune disease include hormones (e.g., insulin and thyroglobulin), glutamic acid decarboxylase, collagen, antibodies, chromatin, nucleoproteins, DNA, RNA, histones, myelin basic protein, proteolipid protein, myosin, P2 protein of peripheral nerve myelin, Rh blood group antigens, gpIIb:IIIa integrin, noncollagenous basement membrane protein, and acetylcholine receptor. The foregoing list is exemplary and is not to be understood to be limiting.

According to this and other aspects of the invention, in one embodiment the therapeutic agent is an immunostimulatory nucleic acid molecule. In one embodiment the immunostimulatory nucleic acid molecule is a CpG nucleic acid molecule. In a particular embodiment the immunostimulatory nucleic acid molecule is a CpG oligonucleotide.

CpG immunostimulatory nucleic acids, including CpG oligonucleotides, are known to stimulate Th1-type immune responses. CpG sequences, while relatively rare in human DNA, are commonly found in the DNA of infectious organisms such as bacteria. The human immune system has apparently evolved to recognize CpG sequences as an early warning sign of infection and to initiate an immediate and powerful immune response against invading pathogens without causing adverse reactions frequently seen with other immune stimulatory agents. Thus CpG-containing nucleic acids, relying on this innate immune defense mechanism, can utilize a unique and natural pathway for immune therapy. The effects of CpG nucleic acids on immune modulation have been described extensively in U.S. patents such as U.S. Pat. No. 6,194,388, U.S. Pat. No. 6,207,646, U.S. Pat. No. 6,239,116 and U.S. Pat. No. 6,218,371, and published international patent applications, such as WO 98/37919, WO 98/52581, WO 98/40100, and WO 99/56755. The entire contents of each of these patents and published patent applications is hereby incorporated by reference.

In one embodiment the immunostimulatory nucleic acid molecule or CpG nucleic acid molecule has a stabilized backbone. As discussed above with reference to the abasic oligonucleotides, the stabilized backbone can in one embodiment include at least one phosphorothioate, phosphorodithioate, alkyl- or arylphosphonate, or alkyl- or arylphosphorothiate linkage. In one embodiment the immunostimulatory nucleic acid molecule or CpG nucleic acid molecule has a phosphorothioate backbone. Other stabilized immunostimulatory nucleic acid molecules or CpG nucleic acid molecules, which are functionally characterized as being relatively resistant to nuclease degradation compared to phosphodiester, are also contemplated by the invention.

According to this and other aspects of the invention, in one embodiment the therapeutic agent is a small molecule. Small molecules include virtually all drugs except for certain biologicals that are macromolecules, e.g., antibodies and recombinant proteins. Nonlimiting lists and examples of drugs, including antimicrobial antibiotics, chemotherapeutic agents, anti-inflammatory agents, agents useful in the treatment of asthma or allergy, are provided below and are embraced within this and all aspects of the invention. In certain embodiments a small molecule is an immunosuppressive drug. Immunosuppressive drugs include, without limitation, cyclosporine, tacrolimus (FK-506), sirolimus (also known as rapamycin), mycophenolate mofetil, azathioprine, corticosteroids (including methylprednisolone and prednisone), and statins (e.g., lovastatin, pravastatin, simvastatin).

In one embodiment the small molecule is an anti-inflammatory drug.

In one embodiment the small molecule is a nucleoside analog useful for treating infection with human immunodeficiency virus. In one embodiment the small molecule is a retroviral protease inhibitor useful for treating infection with human immunodeficiency virus.

In one embodiment the small molecule is an imidazoquinoline. As used herein, an imidazoquinoline includes imidazoquinoline amines, imidazopyridine amines, 6,7-fused cycloalkylimidazopyridine amines, and 1,2 bridged imidazoquinoline amines. These compounds have been described in U.S. Pat. Nos. 4,689,338, 4,929,624, 5,238,944, 5,266,575, 5,268,376, 5,346,905, 5,352,784, 5,389,640, 5,395,937, 5,494,916, 5,482,936, 5,525,612, 6,039,969 and 6,110,929. Particular species of imidazoquinoline agents include 4-amino-α,α-dimethyl-2-ethoxymethyl-1H-imidazo[4,5-c]quinoline-1-ethanol (resiquimod or R-848 or S-28463; WO 02/22125); and 1-(2-methylpropyl)-1H-imidazo[4,5-c]quinoline-4-amine (imiquimod or R-837 or S-26308). Imiquimod is currently used in the topical treatment of warts such as genital and anal warts and has also been tested in the topical treatment of basal cell carcinoma. In one embodiment the small molecule is resiquimod (R-848). In one embodiment the small molecule is imiquimod (R-837).

In one embodiment the small molecule is an inhibitor of immunostimulatory DNA chosen from 9-aminoacridines and 4-aminoquinolines as disclosed in U.S. Pat. Nos. 6,221,882, 6,399,630, 6,479,504, and 6,521,637, the entire contents of which are incorporated herein by reference.

The conjugate can include more than one abasic oligonucleotide, more than one therapeutic agent, or more than one abasic oligonucleotide and more than one therapeutic agent. In one embodiment the conjugate includes a plurality of identical therapeutic agents. In another embodiment the conjugate includes a plurality of non-identical therapeutic agents. In one particular embodiment the conjugate includes a plurality of non-identical therapeutic agents, wherein one therapeutic agent is a ligand for a first TLR and another therapeutic agent is a ligand for a second TLR. In one particular embodiment the conjugate includes a plurality of non-identical therapeutic agents, wherein one therapeutic agent is an agonist for a first TLR and another therapeutic agent is an antagonist for a second TLR. For example, in one embodiment the conjugate includes a plurality of non-identical therapeutic agents, wherein one therapeutic agent is an agonist for TLR7 and another therapeutic agent is an antagonist for TLR8.

In one embodiment according to this and other aspects of the invention, the abasic oligonucleotide and the therapeutic agent are covalently coupled to one another. The covalent coupling can be accomplished directly or indirectly, including through a linker moiety, using any suitable chemical approach. The covalent coupling typically is accomplished as a separate step following synthesis of the abasic oligonucleotide, but in certain embodiments the therapeutic agent can be covalently coupled to the abasic oligonucleotide as part of the synthesis of the abasic oligonucleotide. For example, the therapeutic agent can be cleavably linked to a solid support and provide a terminus upon which the oligonucleotide is synthesized. More typically, however, the therapeutic agent is covalently coupled to the 5′ end or to the 3′ end or to both the 5′ and the 3′ ends of the abasic oligonucleotide. As another example of covalently coupling the therapeutic agent to the abasic oligonucleotide as part of the synthesis of the abasic oligonucleotide, a conjugate that includes an immunostimulatory oligonucleotide as the therapeutic agent can be synthesized in a single integrated, programmed synthesis.

In one embodiment the abasic oligonucleotide is sulfhydril-modified and the therapeutic agent is a protein or polypeptide that is modified with the crosslinker sulfo-maleimidobenzoyl-N-hydroxysuccinamide ester (S-MBS; Pierce). The sulfhydril-modified abasic oligonucleotide is reduced using 50 mM 1,4-dithiothreitiol (DTT)-PBS. Unbound S-MBS and excess DTT are removed by chromatography. An excess of activated abasic oligonucleotide is incubated with linker-modified protein or polypeptide for 2.5 hours and then L-cysteine is added to quench reactive S-MB S. Free abasic oligonucleotides are removed by chromatography, and purified conjugates are analyzed using SDS-PAGE.

The invention in one aspect provides a vaccine including an abasic oligonucleotide 10-40 units long covalently linked to an antigen.

Also provided in one aspect of the invention is a method of increasing antigen uptake by an APC. The method according to this aspect of the invention involves the step of contacting an APC with a composition including a conjugate of an abasic oligonucleotide 10-40 units long and an antigen, in an effective amount to permit antigen uptake by the APC, wherein for a given amount of the antigen, an amount of the antigen taken up by the APC is greater when the APC is contacted with the conjugate than when the APC is contacted with the antigen alone. In one embodiment the APC is a dendritic cell. In order to compare the amount of antigen taken up alone or as a conjugate with the abasic oligonucleotide, in one embodiment equimolar amounts of antigen are contacted, separately and in parallel, with equal numbers of APC. The amount of uptake is measured for each sample and compared. If uptake of antigen by APC contacted with antigen as a conjugate exceeds uptake of antigen by APC contacted with antigen alone, then antigen uptake by APC is said to be increased. Uptake of antigen alone, which serves as a control, can be measured concurrently or otherwise, and use of a concurrent or historical control is embraced by the method.

The method according to this aspect of the invention can be performed in vivo by administering to a subject a composition including a conjugate of an abasic oligonucleotide 10-40 units long and an antigen, in an effective amount to permit antigen uptake by APC of the subject, wherein for a given amount of the antigen administered to the subject, an amount of the antigen taken up by the APC is greater when the subject is administered the conjugate than when the subject is administered the antigen alone. In order to compare the amount of antigen taken up alone or as a conjugate with the abasic oligonucleotide, equimolar amounts of antigen can be administered, separately and on different occasions, to the subject. APC of the subject can be isolated from the subject after administration of the conjugate or after administration of the antigen alone for analysis. The amount of uptake is measured for each sample and compared. If uptake of antigen by APC contacted with antigen as a conjugate exceeds uptake of antigen by APC contacted with antigen alone, then antigen uptake by APC is said to be increased.

The invention in one aspect provides a method of vaccinating a subject. The method according to this aspect involves the step of administering to a subject a composition including a conjugate of an abasic oligonucleotide 10-40 units long and an antigen, in an effective amount to induce an immune response to the antigen in the subject. Induction of an immune response to the antigen can be measured using any suitable method known to the skilled artisan. An immune response to an antigen can be accompanied, for example, by an increased titer of antigen-specific antibody in the serum of the subject. An immune response to an antigen can also be detected by measuring immune cell proliferation, immune cell activation markers, immune cell transcripts, immune cell secretion of cytokines, or immune cell cytolytic activity. This list is exemplary and is not to intended to be limiting. Such measurements can be made and compared using appropriate paired samples, e.g., blood samples, obtained from a subject prior to and following administration of the conjugate to the subject. As is well known in the art, vaccination may be more effective with the administration of appropriately timed booster doses of antigen. Accordingly, the method of vaccination according to this aspect of the invention can include a single administration or more than one administration of antigen-containing conjugate to the subject.

In yet another aspect the invention provides a method of increasing delivery of a TLR signaling agonist to a TLR. The method according to this aspect of the invention involves contacting a cell expressing a TLR with a composition including a conjugate of an abasic oligonucleotide 10-40 units long and a TLR signaling agonist specific for the TLR, in an effective amount to deliver the TLR signaling agonist to the TLR, wherein for a given amount of the TLR signaling agonist contacted with the cell, an amount of TLR signaling agonist delivered to the TLR is greater when the cell is contacted with the conjugate than when the cell is contacted with the TLR signaling agonist alone. The amount of TLR signaling agonist delivered to the TLR can be determined directly or, more commonly, indirectly, for example by measuring a downstream event in immune activation. In one embodiment the amount of TLR signaling agonist delivered to the TLR is measured by measuring expression of a reporter gene that is responsive to a transcription factor or gene product that increases in response to TLR-mediated intracellular signaling. For example, one reporter that can be useful in this aspect of the invention is an NF-κB-luciferase construct. Signaling through a TLR results in generation of NF-κB, which through interaction with an NF-κB-luciferase reporter stimulates expression of enzymatically active luciferase, which can in turn be measured using a luminometer. Comparison can be made between reporter activity associated with contacting a TLR-expressing cell with conjugate versus contacting the cell with TLR signaling agonist alone.

The antigen-containing conjugates of the invention can be used alone or in conjunction with a CpG nucleic acid molecule, e.g., a CpG oligonucleotide. For cross presentation immature DC require enhanced antigen uptake and a maturation signal to prime for major histocompatibility complex (MHC) class I restricted cytotoxic T lymphocyte (CTL) responses in vivo. Thus it has been reported that conjugates of CpG DNA linked to antigen provide DC with both enhanced antigen uptake and the maturation signal. The enhanced antigen uptake is accomplished via receptor-mediated uptake that acts in a sequence-nonspecific manner on nucleic acid, while the maturation signal is provided by the sequence-specific interaction between CpG DNA and TLR9. Accordingly, in one embodiment an antigen-containing conjugate of the invention, which includes antigen and abasic oligonucleotide, can be contacted with a DC or administered to a subject, in conjunction with contacting the DC or administering to the subject a CpG nucleic acid. The conjugate and the CpG nucleic acid may be contacted or administered via the same or different routes. In addition, the conjugate may be contacted or administered before, after, or concurrently with the CpG nucleic acid, provided the desired effect, namely enhanced antigen uptake and DC maturation, is achieved.

When the antigen-containing conjugates of the invention are used alone and in the absence of a DC maturation signal, then DC will have enhanced antigen uptake without maturation, resulting in enhanced antigen presentation in the context of MHC class I without costimulation. Such enhanced antigen presentation without costimulation may result in antigen-specific anergy. This may be useful in any application where it is desirable to promote tolerance, e.g., in the treatment of allergy, autoimmunity, and allograft rejection.

The antigen-containing conjugates of the invention can be used in conjunction with an inhibitory oligonucleotide. This combination retains the conjugate-mediated enhanced uptake of antigen while also providing an inhibitory composition to block a CpG DNA-mediated DC maturation signal, resulting in enhanced antigen presentation in the context of MHC class I without costimulation. As described above, such enhanced antigen presentation without costimulation may result in antigen-specific anergy and may be useful in any application where it is desirable to promote tolerance, e.g., in the treatment of allergy, autoimmunity, and allograft rejection. The conjugate and the inhibitory nucleic acid may be contacted or administered via the same or different routes. In addition, the conjugate may be contacted or administered before, after, or concurrently with the inhibitory nucleic acid, provided the desired effect, namely enhanced antigen uptake and inhibition of DC maturation, is achieved.

The compositions and methods of the invention can be used alone or in conjunction with other agents and methods useful for the treatment of infection. Infection medicaments include but are not limited to anti-bacterial agents, anti-viral agents, anti-fungal agents, and anti-parasitic agents. Phrases such as “anti-infective agent”, “antibiotic”, “anti-bacterial agent”, “anti-viral agent”, “anti-fungal agent”, “anti-parasitic agent” and “parasiticide” have well-established meanings to those of ordinary skill in the art and are defined in standard medical texts. Briefly, anti-bacterial agents kill or inhibit bacteria, and include antibiotics as well as other synthetic or natural compounds having similar functions. Anti-viral agents can be isolated from natural sources or synthesized and are useful for killing or inhibiting viruses. Anti-fungal agents are used to treat superficial fungal infections as well as opportunistic and primary systemic fungal infections. Anti-parasite agents kill or inhibit parasites. Many antibiotics are low molecular weight molecules which are produced as secondary metabolites by cells, such as microorganisms. In general, antibiotics interfere with one or more functions or structures which are specific for the microorganism and which are not present in host cells.

One of the problems with anti-infective therapies is the side effects occurring in the host that is treated with the anti-infective agent. For instance, many anti-infectious agents can kill or inhibit a broad spectrum of microorganisms and are not specific for a particular type of species. Treatment with these types of anti-infectious agents results in the killing of the normal microbial flora living in the host, as well as the infectious microorganism. The loss of the microbial flora can lead to disease complications and predispose the host to infection by other pathogens, since the microbial flora compete with and function as barriers to infectious pathogens. Other side effects may arise as a result of specific or non-specific effects of these chemical entities on non-microbial cells or tissues of the host.

Another problem with widespread use of anti-infectants is the development of antibiotic-resistant strains of microorganisms. Already, vancomycin-resistant Enterococci, penicillin-resistant Pneumococci, multi-resistant S. aureus, and multi-resistant tuberculosis strains have developed and are becoming major clinical problems. Widespread use of anti-infectants will likely produce many antibiotic-resistant strains of bacteria. As a result, new anti-infective strategies will be required to combat these microorganisms.

Antibacterial antibiotics which are effective for killing or inhibiting a wide range of bacteria are referred to as broad-spectrum antibiotics. Other types of antibacterial antibiotics are predominantly effective against the bacteria of the class gram-positive or gram-negative. These types of antibiotics are referred to as narrow-spectrum antibiotics. Other antibiotics which are effective against a single organism or disease and not against other types of bacteria, are referred to as limited-spectrum antibiotics.

Anti-bacterial agents are sometimes classified based on their primary mode of action. In general, anti-bacterial agents are cell wall synthesis inhibitors, cell membrane inhibitors, protein synthesis inhibitors, nucleic acid synthesis or functional inhibitors, and competitive inhibitors. Cell wall synthesis inhibitors inhibit a step in the process of cell wall synthesis, and in general in the synthesis of bacterial peptidoglycan. Cell wall synthesis inhibitors include β-lactam antibiotics, natural penicillins, semi-synthetic penicillins, ampicillin, clavulanic acid, cephalolsporins, and bacitracin.

The β-lactams are antibiotics containing a four-membered β-lactam ring which inhibits the last step of peptidoglycan synthesis. β-lactam antibiotics can be synthesized or natural. The β-lactam antibiotics produced by penicillium are the natural penicillins, such as penicillin G or penicillin V. These are produced by fermentation of Penicillium chrysogenum. The natural penicillins have a narrow spectrum of activity and are generally effective against Streptococcus, Gonococcus, and Staphylococcus. Other types of natural penicillins, which are also effective against gram-positive bacteria, include penicillins F, X, K, and O.

Semi-synthetic penicillins are generally modifications of the molecule 6-aminopenicillanic acid produced by a mold. The 6-aminopenicillanic acid can be modified by addition of side chains which produce penicillins having broader spectrums of activity than natural penicillins or various other advantageous properties. Some types of semi-synthetic penicillins have broad spectrums against gram-positive and gram-negative bacteria, but are inactivated by penicillinase. These semi-synthetic penicillins include ampicillin, carbenicillin, oxacillin, azlocillin, mezlocillin, and piperacillin. Other types of semi-synthetic penicillins have narrower activities against gram-positive bacteria, but have developed properties such that they are not inactivated by penicillinase. These include, for instance, methicillin, dicloxacillin, and nafcillin. Some of the broad spectrum semi-synthetic penicillins can be used in combination with β-lactamase inhibitors, such as clavulanic acids and sulbactam. The β-lactamase inhibitors do not have anti-microbial action but they function to inhibit penicillinase, thus protecting the semi-synthetic penicillin from degradation.

One of the serious side effects associated with penicillins, both natural and semi-synthetic, is penicillin allergy. Penicillin allergies are very serious and can cause death rapidly. In a subject that is allergic to penicillin, the β-lactam molecule will attach to a serum protein which initiates an IgE-mediated inflammatory response. The inflammatory response leads to anaphylaxis and possibly death.

Another type of β-lactam antibiotic is the cephalolsporins. They are sensitive to degradation by bacterial β-lactamases, and thus, are not always effective alone. Cephalolsporins, however, are resistant to penicillinase. They are effective against a variety of gram-positive and gram-negative bacteria. Cephalolsporins include, but are not limited to, cephalothin, cephapirin, cephalexin, cefamandole, cefaclor, cefazolin, cefuroxine, cefoxitin, cefotaxime, cefsulodin, cefetamet, cefixime, ceftriaxone, cefoperazone, ceftazidine, and moxalactam.

Bacitracin is another class of antibiotics which inhibit cell wall synthesis, by inhibiting the release of muropeptide subunits or peptidoglycan from the molecule that delivers the subunit to the outside of the membrane. Although bacitracin is effective against gram-positive bacteria, its use is limited in general to topical administration because of its high toxicity.

Carbapenems are another broad-spectrum β-lactam antibiotic, which is capable of inhibiting cell wall synthesis. Examples of carbapenems include, but are not limited to, imipenems. Monobactams are also broad-spectrum β-lactam antibiotics, and include, euztreonam. An antibiotic produced by Streptomyces, vancomycin, is also effective against gram-positive bacteria by inhibiting cell membrane synthesis.

Another class of anti-bacterial agents is the anti-bacterial agents that are cell membrane inhibitors. These compounds disorganize the structure or inhibit the function of bacterial membranes. One problem with anti-bacterial agents that are cell membrane inhibitors is that they can produce effects in eukaryotic cells as well as bacteria because of the similarities in phospholipids in bacterial and eukaryotic membranes. Thus these compounds are rarely specific enough to permit these compounds to be used systemically and prevent the use of high doses for local administration.

One clinically useful cell membrane inhibitor is Polymyxin. Polymyxins interfere with membrane function by binding to membrane phospholipids. Polymyxin is effective mainly against Gram-negative bacteria and is generally used in severe Pseudomonas infections or Pseudomonas infections that are resistant to less toxic antibiotics. The severe side effects associated with systemic administration of this compound include damage to the kidney and other organs.

Other cell membrane inhibitors include Amphotericin B and Nystatin which are anti-fungal agents used predominantly in the treatment of systemic fungal infections and Candida yeast infections. Imidazoles are another class of antibiotic that is a cell membrane inhibitor. Imidazoles are used as anti-bacterial agents as well as anti-fungal agents, e.g., used for treatment of yeast infections, dermatophytic infections, and systemic fungal infections. Imidazoles include but are not limited to clotrimazole, miconazole, ketoconazole, itraconazole, and fluconazole.

Many anti-bacterial agents are protein synthesis inhibitors. These compounds prevent bacteria from synthesizing structural proteins and enzymes and thus cause inhibition of bacterial cell growth or function or cell death. In general these compounds interfere with the processes of transcription or translation. Anti-bacterial agents that block transcription include but are not limited to Rifampins and Ethambutol. Rifampins, which inhibit the enzyme RNA polymerase, have a broad spectrum activity and are effective against gram-positive and gram-negative bacteria as well as Mycobacterium tuberculosis. Ethambutol is effective against Mycobacterium tuberculosis.

Anti-bacterial agents which block translation interfere with bacterial ribosomes to prevent mRNA from being translated into proteins. In general this class of compounds includes but is not limited to tetracyclines, chloramphenicol, the macrolides (e.g., erythromycin) and the aminoglycosides (e.g., streptomycin).

The aminoglycosides are a class of antibiotics which are produced by the bacterium Streptomyces, such as, for instance streptomycin, kanamycin, tobramycin, amikacin, and gentamicin. Aminoglycosides have been used against a wide variety of bacterial infections caused by Gram-positive and Gram-negative bacteria. Streptomycin has been used extensively as a primary drug in the treatment of tuberculosis. Gentamicin is used against many strains of Gram-positive and Gram-negative bacteria, including Pseudomonas infections, especially in combination with Tobramycin. Kanamycin is used against many Gram-positive bacteria, including penicillin-resistant Staphylococci. One side effect of aminoglycosides that has limited their use clinically is that at dosages which are essential for efficacy, prolonged use has been shown to impair kidney function and cause damage to the auditory nerves leading to deafness.

Another type of translation inhibitor anti-bacterial agent is the tetracyclines. The tetracyclines are a class of antibiotics that are broad-spectrum and are effective against a variety of gram-positive and gram-negative bacteria. Examples of tetracyclines include tetracycline, minocycline, doxycycline, and chlortetracycline. They are important for the treatment of many types of bacteria but are particularly important in the treatment of Lyme disease. As a result of their low toxicity and minimal direct side effects, the tetracyclines have been overused and misused by the medical community, leading to problems. For instance, their overuse has led to widespread development of resistance.

Anti-bacterial agents such as the macrolides bind reversibly to the 50 S ribosomal subunit and inhibit elongation of the protein by peptidyl transferase or prevent the release of uncharged tRNA from the bacterial ribosome or both. These compounds include erythromycin, roxithromycin, clarithromycin, oleandomycin, and azithromycin. Erythromycin is active against most Gram-positive bacteria, Neisseria, Legionella and Haemophilus, but not against the Enterobacteriaceae. Lincomycin and clindamycin, which block peptide bond formation during protein synthesis, are used against gram-positive bacteria.

Another type of translation inhibitor is chloramphenicol. Chloramphenicol binds the 70 S ribosome inhibiting the bacterial enzyme peptidyl transferase thereby preventing the growth of the polypeptide chain during protein synthesis. One serious side effect associated with chloramphenicol is aplastic anemia. Aplastic anemia develops at doses of chloramphenicol which are effective for treating bacteria in a small proportion ( 1/50,000) of patients. Chloramphenicol which was once a highly prescribed antibiotic is now seldom uses as a result of the deaths from anemia. Because of its effectiveness it is still used in life-threatening situations (e.g., typhoid fever).

Some anti-bacterial agents disrupt nucleic acid synthesis or function, e.g., bind to DNA or RNA so that their messages cannot be read. These include but are not limited to quinolones and co-trimoxazole, both synthetic chemicals and rifamycins, a natural or semi-synthetic chemical. The quinolones block bacterial DNA replication by inhibiting the DNA gyrase, the enzyme needed by bacteria to produce their circular DNA. They are broad spectrum and examples include norfloxacin, ciprofloxacin, enoxacin, nalidixic acid and temafloxacin. Nalidixic acid is a bactericidal agent that binds to the DNA gyrase enzyme (topoisomerase) which is essential for DNA replication and allows supercoils to be relaxed and reformed, inhibiting DNA gyrase activity. The main use of nalidixic acid is in treatment of lower urinary tract infections (UTI) because it is effective against several types of Gram-negative bacteria such as E. coli, Enterobacter aerogenes, K. pneumoniae and Proteus species which are common causes of UTI. Co-trimoxazole is a combination of sulfamethoxazole and trimethoprim, which blocks the bacterial synthesis of folic acid needed to make DNA nucleotides. Rifainpicin is a derivative of rifamycin that is active against Gram-positive bacteria (including Mycobacterium tuberculosis and meningitis caused by Neisseria meningitides) and some Gram-negative bacteria. Rifampicin binds to the beta subunit of the polymerase and blocks the addition of the first nucleotide which is necessary to activate the polymerase, thereby blocking mRNA synthesis.

Another class of anti-bacterial agents is compounds that function as competitive inhibitors of bacterial enzymes. The competitive inhibitors are mostly all structurally similar to a bacterial growth factor and compete for binding but do not perform the metabolic function in the cell. These compounds include sulfonamides and chemically modified forms of sulfanilamide which have even higher and broader antibacterial activity. The sulfonamides (e.g., gantrisin and trimethoprim) are useful for the treatment of Streptococcus pneumoniae, beta-hemolytic streptococci and E. coli, and have been used in the treatment of uncomplicated UTI caused by E. coli, and in the treatment of meningococcal meningitis.

Anti-viral agents are compounds which prevent infection of cells by viruses or replication of the virus within the cell. There are many fewer antiviral drugs than antibacterial drugs because the process of viral replication is so closely related to DNA replication within the host cell, that non-specific antiviral agents would often be toxic to the host. There are several stages within the process of viral infection which can be blocked or inhibited by antiviral agents. These stages include, attachment of the virus to the host cell (inumunoglobulin or binding peptides), uncoating of the virus (e.g. amantadine), synthesis or translation of viral mRNA (e.g. interferon), replication of viral RNA or DNA (e.g. nucleoside analogues), maturation of new virus proteins (e.g. protease inhibitors), and budding and release of the virus.

Another category of anti-viral agents are nucleoside analogues. Nucleoside analogues are synthetic compounds which are similar to nucleosides, but which have an incomplete or abnormal deoxyribose or ribose group. Once the nucleoside analogues are in the cell, they are phosphorylated, producing the triphosphate form which competes with normal nucleotides for incorporation into the viral DNA or RNA. Once the triphosphate form of the nucleoside analogue is incorporated into the growing nucleic acid chain, it causes irreversible association with the viral polymerase and thus chain termination. Nucleoside analogues include, but are not limited to, acyclovir (used for the treatment of herpes simplex virus and varicella-zoster virus), gancyclovir (useful for the treatment of cytomegalovirus), idoxuridine, ribavirin (useful for the treatment of respiratory syncitial virus), dideoxyinosine, dideoxycytidine, and zidovudine (azidothymidine).

Another class of anti-viral agents includes cytokines such as interferons. The interferons are cytokines which are secreted by virus-infected cells as well as immune cells. The interferons function by binding to specific receptors on cells adjacent to the infected cells, causing the change in the cell which protects it from infection by the virus. α and β-interferon also induce the expression of Class I and Class II MHC molecules on the surface of infected cells, resulting in increased antigen presentation for host immune cell recognition. α and β-interferons are available as recombinant forms and have been used for the treatment of chronic hepatitis B and C infection. At the dosages which are effective for anti-viral therapy, interferons have severe side effects such as fever, malaise and weight loss.

Immunoglobulin therapy is used for the prevention of viral infection. Immunoglobulin therapy for viral infections is different from bacterial infections, because rather than being antigen-specific, the immunoglobulin therapy functions by binding to extracellular virions and preventing them from attaching to and entering cells which are susceptible to the viral infection. The therapy is useful for the prevention of viral infection for the period of time that the antibodies are present in the host. In general there are two types of immunoglobulin therapies, normal immune globulin therapy and hyper-immune globulin therapy. Normal immune globulin therapy utilizes a antibody product which is prepared from the serum of normal blood donors and pooled. This pooled product contains low titers of antibody to a wide range of human viruses, such as hepatitis A, parvovirus, enterovirus (especially in neonates). Hyper-immune globulin therapy utilizes antibodies which are prepared from the serum of individuals who have high titers of an antibody to a particular virus. Those antibodies are then used against a specific virus. Examples of hyper-immune globulins include zoster immune globulin (useful for the prevention of varicella in immunocompromised children and neonates), human rabies immune globulin (useful in the post-exposure prophylaxis of a subject bitten by a rabid animal), hepatitis B immune globulin (useful in the prevention of hepatitis B virus, especially in a subject exposed to the virus), and RSV immune globulin (useful in the treatment of respiratory syncitial virus infections).

Anti-fungal agents are useful for the treatment and prevention of infective fungi. Anti-fungal agents are sometimes classified by their mechanism of action. Some anti-fungal agents function as cell wall inhibitors by inhibiting glucose synthase. These include, but are not limited to, basiungin/ECB. Other anti-fungal agents function by destabilizing membrane integrity. These include, but are not limited to, imidazoles, such as clotrimazole, sertaconzole, fluconazole, itraconazole, ketoconazole, miconazole, and voriconacole, as well as FK 463, amphotericin B, BAY 38-9502, MK 991, pradimicin, UK 292, butenafine, and terbinafine. Other anti-fungal agents function by breaking down chitin (e.g., chitinase) or immunosuppression (501 cream).

Parasiticides are agents that kill parasites directly. Such compounds are known in the art and are generally commercially available. Examples of parasiticides useful for human administration include but are not limited to albendazole, amphotericin B, benznidazole, bithionol, chloroquine HCl, chloroquine phosphate, clindamycin, dehydroemetine, diethylcarbamazine, diloxanide furoate, eflomithine, furazolidaone, glucocorticoids, halofantrine, iodoquinol, ivermectin, mebendazole, mefloquine, meglumine antimoniate, melarsoprol, metrifonate, metronidazole, niclosamide, nifurtimox, oxamniquine, paromomycin, pentamidine isethionate, piperazine, praziquantel, primaquine phosphate, proguanil, pyrantel pamoate, pyrimethamine-sulfonamides, pyrimethamine-sulfadoxine, quinacrine HCl, quinine sulfate, quinidine gluconate, spiramycin, stibogluconate sodium (sodium antimony gluconate), suramin, tetracycline, doxycycline, thiabendazole, timidazole, trimethroprim-sulfamethoxazole, and tryparsamide.

The compositions and methods of the invention can be used alone or in conjunction with other agents and methods useful for the treatment of cancer. Cancer is currently treated using a variety of modalities including surgery, radiation therapy, and chemotherapy. The choice of treatment modality will depend upon the type, location and dissemination of the cancer. For example, surgery and radiation therapy may be more appropriate in the case of solid, well-defined tumor masses and less practical in the case of non-solid tumor cancers such as leukemia and lymphoma. One of the advantages of surgery and radiation therapy is the ability to control to some extent the impact of the therapy, and thus to limit the toxicity to normal tissues in the body. However, surgery and radiation therapy are often followed by chemotherapy to guard against any remaining or radio-resistant cancer cells. Chemotherapy is also appropriate treatment for disseminated cancers such as leukemia and lymphoma as well as metastases.

Chemotherapy refers to therapy using chemical and/or biological agents to attack cancer cells. Unlike localized surgery or radiation, chemotherapy is generally administered in a systemic fashion and thus toxicity to normal tissues is a major concern. Because many chemotherapy agents target cancer cells based on their proliferative profiles, tissues such as the gastrointestinal tract and the bone marrow, which are normally proliferative, are also susceptible to the effects of the chemotherapy. One of the major side effects of chemotherapy is myelosuppression (including anemia, neutropenia and thrombocytopenia) which results from the death of normal hemopoietic precursors.

Many chemotherapeutic agents have been developed for the treatment of cancer. Not all tumors, however, respond to chemotherapeutic agents and others although initially responsive to chemotherapeutic agents may develop resistance. As a result, the search for effective anti-cancer drugs has intensified in an effort to find even more effective agents with less non-specific toxicity.

Chemotherapeutic agents include 4′-Deoxyoxorubicin, 5-Fluorouracil, 9-AC, AD 32/Valrubicin, Adriamycin, AG3340, AG3433, alkylating agents such as melphelan and cyclophosphamide, Aminoglutethimide, Amsacrine (m-AMSA), Asparaginase, Azacitidine, Aziridine, Batimastat, BAY 12-9566, BB2516/Marmistat, BCH-4556, Bleomycin, BMS-182751/oral platinum, Busulfan, Caelyx/liposomal doxorubicin, Caetyx/liposomal doxorubicin, Campto/Levamisole, Camptosar/Irinotecan, Carboplatin, carmustine, CDK4 and CDK2 inhibitors, CDP 845, Chlorambucil, CI-994, cisplatin, CP-358 (774)/EGFR, CP-609 (754)/RAS oncogene inhibitor, CS-682, Cyclopax/oral paclitaxel, Cytarabine HCl, D2163, D4809/Dexifosamide, Dacarbazine, Dactinomycin, Daunorubicin HC1, DepoCyt, Doxil/liposomal doxorubicin, Doxorubicin, DX8951f, E7070, Eniluracil/776C85/5FU enhancer, Ergamisol/Levamisole, Erythropoietin, Estramustine phosphate sodium, Etoposide (VP16-213), Evacet/liposomal doxorubicin, Farnesyl transferase inhibitor, FK 317, Floxuridine, Fludara/Fludarabine, Fluorouracil (5-FU), Flutamide, Fragyline, Furtulon/Doxifluridine, Gemzar/Gemcitabine, Glamolec, Hexamethylmelamine (HMM), HMR 1275/Flavopiridol, Hycamtin/Topotecan, Hydroxyurea (hydroxycarbamide), Ifes/Mesnex/Ifosamide, Ifosfamide, Incel/VX-710, Interferon Alfa-2a, Interferon Alfa-2b, Interleukin 2, IS1641, Lemonal DP 2202, Leuprolide acetate (LHR1H-releasing factor analogue), Leustatin/Cladribine, Lomustine (CCNU), LU 103793/Dolastain, LU 79553/Bis-Naphtalimide, LY264618/Lometexol, Mechlorethamine HCl (nitrogen mustard), Meglamine GLA, Mercaptopurine, Mesna, Metaret/Suramin, Metastron/strontium derivative, Methotrexate, Mitoguazone (methyl-GAG; methyl glyoxal bis-guanylhydrazone; MGBG), Mitomycin C, Mitotane (o.p′-DDD), Mitoxantrone HCl, MMI270, MMP, MTA/LY231514, nitrosoureas, non-sugar containing chloroethylnitrosoureas, Novantrone/Mitroxantrone, Octreotide, ODN 698, Oral Taxoid, Paraplatin/Carboplatin, PARP inhibitors, Paxex/Paclitaxel, PD183805, Pentostatin (2′ deoxycoformycin), Pharmarubicin/Epirubicin, Picibanil/OK-432, PKC412, Plantinol/cisplatin, Plicamycin, Procarbazine HC1, prodrug of guanine arabinoside, RAS farnesyl transferase inhibitor, Semustine (methyl-CCNU), SPU-077/Cisplatin, Streptozocin, TA 2516/Marmistat, Tamoxifen citrate, Taxane Analog, Taxol, Taxol/Paclitaxel, Taxotere/Docetaxel, Temodal/Temozolomide, Teniposide (VM-26), Thioguanine, Thiotepa, TNP-470, Tumodex/Ralitrexed, UFT(Tegafur/Uracil), Valrubicin, Valspodar/PSC833, Vepeside/Etoposide, Vinblastine sulfate, Vincristine, Vindesine sulfate, Vumon/Teniposide, VX-853, Xeload/Capecitabine, Yewtaxan/Paclitaxel, YM 116, ZD 0473/Anormed, ZD 9331, ZD0101, and ZD1839, but are not so limited.

Cancer medicaments function in a variety of ways. Some cancer medicaments work by targeting physiological mechanisms that are specific to tumor cells. Examples include the targeting of specific genes and their gene products (i.e., proteins primarily) which are mutated in cancers. Such genes include but are not limited to oncogenes (e.g., Ras, Her2, bcl-2), tumor suppressor genes (e.g., EGF, p53, Rb), and cell cycle targets (e.g., CDK4, p21, telomerase). Cancer medicaments can alternately target signal transduction pathways and molecular mechanisms which are altered in cancer cells. Targeting of cancer cells via the epitopes expressed on their cell surface is accomplished through the use of monoclonal antibodies. This latter type of cancer medicament is generally referred to herein as immunotherapy.

Other cancer medicaments target cells other than cancer cells. For example, some medicaments prime the immune system to attack tumor cells (i.e., cancer vaccines). Still other medicaments, called angiogenesis inhibitors, function by attacking the blood supply of solid tumors. Since most malignant cancers are able to metastasize (i.e., exit the primary tumor site and seed a another site, thereby forming a secondary tumor), medicaments that impede this metastasis are also useful in the treatment of cancer. Angiogenic mediators include basic FGF, VEGF, angiopoietins, angiostatin, endostatin, TNF-α, TNP-470, thrombospondin-1, platelet factor 4, CAI, and certain members of the integrin family of proteins. One category of this type of medicament is a metalloproteinase inhibitor, which inhibits the enzymes used by the cancer cells to exit the primary tumor site and extravasate into another tissue.

Some cancer cells are antigenic and thus can be targeted by the immune system. In one aspect, the combined administration of abasic oligonucleotide and cancer medicaments, particularly those which are classified as cancer immunotherapies, is useful for stimulating a specific immune response against a cancer antigen.

The theory of immune surveillance is that a prime function of the immune system is to detect and eliminate neoplastic cells before a tumor forms. A basic principle of this theory is that cancer cells are antigenically different from normal cells and thus elicit immune reactions that are similar to those that cause rejection of immunologically incompatible allografts. Studies have confirmed that tumor cells differ, either qualitatively or quantitatively, in their expression of antigens. For example, “tumor-specific antigens” are antigens that are specifically associated with tumor cells but not normal cells. Examples of tumor-specific antigens are viral antigens in tumors induced by DNA or RNA viruses. “Tumor-associated” antigens are present in both tumor cells and normal cells but are present in a different quantity or a different form in tumor cells. Examples of such antigens are oncofetal antigens (e.g., carcinoembryonic antigen), differentiation antigens (e.g., T and Tn antigens), and oncogene products (e.g., HER/neu).

Different types of cells that can kill tumor targets in vitro and in vivo have been identified: natural killer (NK) cells, cytolytic T lymphocytes (CTLs), lymphokine-activated killer (LAK) cells, and activated macrophages. NK cells can kill tumor cells without having been previously sensitized to specific antigens, and the activity does not require the presence of class I antigens encoded by the major histocompatibility complex (MHC) on target cells. NK cells are thought to participate in the control of nascent tumors and in the control of metastatic growth. In contrast to NK cells, CTLs can kill tumor cells only after they have been sensitized to tumor antigens and when the target antigen is expressed on the tumor cells that also express MHC class I. CTLs are thought to be effector cells in the rejection of transplanted tissues and of tumors caused by DNA viruses. LAK cells are a subset of null lymphocytes distinct from the NK and CTL populations. Activated macrophages can kill tumor cells in a manner that is neither antigen-dependent nor MHC-restricted. Activated macrophages are thought to decrease the growth rate of the tumors they infiltrate. In vitro assays have identified other immune mechanisms such as antibody-dependent, cell-mediated cytotoxic reactions and lysis by antibody plus complement. However, these immune effector mechanisms are thought to be less important in vivo than the function of macrophages and NK, CTL, and LAK cells (for review see Piessens W F et al. “Tumor Immunology”, In: Scientific American Medicine, Vol. 2, Scientific American Books, N.Y., pp. 1-13, 1996).

The goal of immunotherapy is to augment a patient's immune response to an established tumor. One method of immunotherapy includes the use of adjuvants. Adjuvant substances derived from microorganisms, such as bacillus Calmette-Guérin (BCG), heighten the immune response and enhance resistance to tumors in animals.

Immunotherapeutic agents are medicaments which derive from antibodies or antibody fragments which specifically bind or recognize a cancer antigen. Antibody-based immunotherapies may function by binding to the cell surface of a cancer cell and thereby stimulating the endogenous immune system to attack the cancer cell. Another way in which antibody-based therapy functions is as a delivery system for the specific targeting of toxic substances to cancer cells. Antibodies are usually conjugated to toxins such as ricin (e.g., from castor beans), calicheamicin and maytansinoids; to radioactive isotopes such as Iodine-131 and Yttrium-90; to chemotherapeutic agents (as described herein); or to biological response modifiers. In this way, the toxic substances can be concentrated in the region of the cancer and non-specific toxicity to normal cells can be minimized. In addition to the use of antibodies which are specific for cancer antigens, antibodies which bind to vasculature, such as those which bind to endothelial cells, are also useful in the invention. This is because solid tumors generally are dependent upon newly formed blood vessels to survive, and thus most tumors are capable of recruiting and stimulating the growth of new blood vessels. As a result, one strategy of many cancer medicaments is to attack the blood vessels feeding a tumor and/or the connective tissues (or stroma) supporting such blood vessels.

Cancer vaccines are medicaments which are intended to stimulate an endogenous immune response against cancer cells. Currently produced vaccines predominantly activate the humoral immune system (i.e., the antibody-dependent immune response). Other vaccines currently in development are focused on activating the cell-mediated immune system including cytotoxic T lymphocytes which are capable of killing tumor cells. Cancer vaccines generally enhance the presentation of cancer antigens to both antigen-presenting cells (e.g., macrophages and dendritic cells) and/or to other immune cells such as T cells, B cells, and NK cells.

Although cancer vaccines may take one of several forms, as discussed infra, their purpose is to deliver cancer antigens and/or cancer associated antigens to antigen-presenting cells (APC) in order to facilitate the endogenous processing of such antigens by APC and the ultimate presentation of antigen presentation on the cell surface in the context of MHC class I molecules. One form of cancer vaccine is a whole cell vaccine which is a preparation of cancer cells which have been removed from a subject, treated ex vivo and then reintroduced as whole cells in the subject. Lysates of tumor cells can also be used as cancer vaccines to elicit an immune response. Another form cancer vaccine is a peptide vaccine which uses cancer-specific or cancer-associated small proteins to activate T cells. Cancer-associated proteins are proteins which are not exclusively expressed by cancer cells (i.e., other normal cells may still express these antigens). However, the expression of cancer-associated antigens is generally consistently upregulated with cancers of a particular type. Other cancer vaccines include ganglioside vaccines, heat-shock protein vaccines, viral and bacterial vaccines, and nucleic acid vaccines.

Yet another form of cancer vaccine is a dendritic cell vaccine which includes whole dendritic cells which have been exposed to a cancer antigen or a cancer-associated antigen in vitro. Lysates or membrane fractions of dendritic cells may also be used as cancer vaccines. Dendritic cell vaccines are able to activate APCs directly. A dendritic cell is a professional APC. Dendritic cells form a link between the innate and the acquired immune system by presenting antigens and through their expression of pattern recognition receptors which detect microbial molecules like lipopolysaccharide (LPS) in their local environment. Dendritic cells efficiently internalize, process, and present soluble specific antigen to which they are exposed. The process of internalizing and presenting antigen causes rapid upregulation of the expression of MHC and costimulatory molecules, the production of cytokines, and migration toward lymphatic organs where they are believed to be involved in the activation of T cells.

As used herein, chemotherapeutic agents embrace all other forms of cancer medicaments which do not fall into the categories of immunotherapeutic agents or cancer vaccines. Chemotherapeutic agents as used herein encompass both chemical and biological agents. These agents function to inhibit a cellular activity upon which the cancer cell is dependent for continued survival. Categories of chemotherapeutic agents include alkylating/alkaloid agents, antimetabolites, hormones or hormone analogs, and miscellaneous antineoplastic drugs. Most if not all of these agents are directly toxic to cancer cells and do not require immune stimulation.

The compositions and methods of the invention can be used alone or in conjunction with other agents and methods useful in the treatment of allergy and asthma. An “asthma/allergy medicament” as used herein is a composition of matter which reduces the symptoms of, prevents the development of, or inhibits an asthmatic episode or allergic reaction. Various types of medicaments for the treatment of asthma and allergy are described in the Guidelines For The Diagnosis and Management of Asthma, Expert Panel Report 2, NIH Publication No. 97/4051, Jul. 19, 1997, the entire contents of which are incorporated herein by reference. The summary of the medicaments as described in the NIH publication is presented below. In most embodiments the asthma/allergy medicament is useful to some degree for treating both asthma and allergy.

Medications for the treatment of asthma are generally separated into two categories, quick-relief medications and long-term control medications. Asthma patients take the long-term control medications on a daily basis to achieve and maintain control of persistent asthma. Long-term control medications include anti-inflammatory agents such as corticosteroids, chromolyn sodium and nedocromil; long-acting bronchodilators, such as long-acting β₂-agonists and methylxanthines; and leukotriene modifiers. The quick-relief medications include short-acting β₂ agonists, anticholinergics, and systemic corticosteroids. There are many side effects associated with each of these drugs and none of the drugs alone or in combination is capable of preventing or completely treating asthma.

Asthma medicaments include, but are not limited, PDE-4 inhibitors, bronchodilator/beta-2 agonists, K+ channel openers, VLA-4 antagonists, neurokin antagonists, thromboxane A2 (TXA2) synthesis inhibitors, xanthines, arachidonic acid antagonists, 5 lipoxygenase inhibitors, TXA2 receptor antagonists, TXA2 antagonists, inhibitor of 5-lipox activation proteins, and protease inhibitors.

Bronchodilator/β₂ agonists are a class of compounds which cause bronchodilation or smooth muscle relaxation. Bronchodilator/β₂ agonists include, but are not limited to, salmeterol, salbutamol, albuterol, terbutaline, D2522/formoterol, fenoterol, bitolterol, pirbuterol methylxanthines and orciprenaline. Long-acting β₂ agonists and bronchodilators are compounds which are used for long-term prevention of symptoms in addition to the anti-inflammatory therapies. Long-acting β₂ agonists include, but are not limited to, salmeterol and albuterol. These compounds are usually used in combination with corticosteroids and generally are not used without any inflammatory therapy. They have been associated with side effects such as tachycardia, skeletal muscle tremor, hypokalemia, and prolongation of QTc interval in overdose.

Methylxanthines, including for instance theophylline, have been used for long-term control and prevention of symptoms. These compounds cause bronchodilation resulting from phosphodiesterase inhibition and likely adenosine antagonism. Dose-related acute toxicities are a particular problem with these types of compounds. As a result, routine serum concentration must be monitored in order to account for the toxicity and narrow therapeutic range arising from individual differences in metabolic clearance. Side effects include tachycardia, tachyarrhythmias, nausea and vomiting, central nervous system stimulation, headache, seizures, hematemesis, hyperglycemia and hypokalemia. Short-acting β₂ agonists include, but are not limited to, albuterol, bitolterol, pirbuterol, and terbutaline. Some of the adverse effects associated with the administration of short-acting β2 agonists include tachycardia, skeletal muscle tremor, hypokalemia, increased lactic acid, headache, and hyperglycemia.

Conventional methods for treating or preventing allergy have involved the use of anti-histamines or desensitization therapies. Anti-histamines and other drugs which block the effects of chemical mediators of the allergic reaction help to regulate the severity of the allergic symptoms but do not prevent the allergic reaction and have no effect on subsequent allergic responses. Desensitization therapies are performed by giving small doses of an allergen, usually by injection under the skin, in order to induce an IgG-type response against the allergen. The presence of IgG antibody helps to neutralize the production of mediators resulting from the induction of IgE antibodies, it is believed. Initially, the subject is treated with a very low dose of the allergen to avoid inducing a severe reaction and the dose is slowly increased. This type of therapy is dangerous because the subject is actually administered the compounds which cause the allergic response and severe allergic reactions can result.

Allergy medicaments include, but are not limited to, anti-histamines, steroids, and prostaglandin inducers. Anti-histamines are compounds which counteract histamine released by mast cells or basophils. These compounds are well known in the art and commonly used for the treatment of allergy. Anti-histamines include, but are not limited to, astemizole, azelastine, betatastine, buclizine, ceterizine, cetirizine analogues, CS 560, desloratadine, ebastine, epinastine, fexofenadine, HSR 609, levocabastine, loratidine, mizolastine, norastemizole, terfenadine, and tranilast.

Prostaglandin inducers are compounds which induce prostaglandin activity. Prostaglandins function by regulating smooth muscle relaxation. Prostaglandin inducers include, but are not limited to, S-5751.

The asthma/allergy medicaments also include steroids and immunomodulators. The steroids include, but are not limited to, beclomethasone, fluticasone, triamcinolone, corticosteroids, and budesonide.

Corticosteroids include, but are not limited to, beclomethasome dipropionate, budesonide, flunisolide, fluticaosone propionate, and triamcinolone acetonide. Although dexamethasone is a corticosteroid having anti-inflammatory action, it is not regularly used for the treatment of asthma/allergy in an inhaled form because it is highly absorbed and it has long-term suppressive side effects at an effective dose. Dexamethasone, however, can be used according to the invention for the treating of asthma/allergy because when administered in combination with nucleic acids of the invention it can be administered at a low dose to reduce the side effects. Some of the side effects associated with corticosteroid include cough, dysphonia, oral thrush (candidiasis), and in higher doses, systemic effects, such as adrenal suppression, osteoporosis, growth suppression, skin thinning and easy bruising. Barnes & Peterson (1993) Am Rev Respir Dis 148:S1-S26; and Kamada A K et al. (1996) Am J Respir Crit. Care Med 153:1739-48.

Systemic corticosteroids include, but are not limited to, methylprednisolone, prednisolone and prednisone. Cortosteroids are associated with reversible abnormalities in glucose metabolism, increased appetite, fluid retention, weight gain, mood alteration, hypertension, peptic ulcer, and aseptic necrosis of bone. These compounds are useful for short-term (3-10 days) prevention of the inflammatory reaction in inadequately controlled persistent asthma. They also function in a long-term prevention of symptoms in severe persistent asthma to suppress and control and actually reverse inflammation. Some side effects associated with longer term use include adrenal axis suppression, growth suppression, dermal thinning, hypertension, diabetes, Cushing's syndrome, cataracts, muscle weakness, and in rare instances, impaired immune function. It is recommended that these types of compounds be used at their lowest effective dose (guidelines for the diagnosis and management of asthma; expert panel report to; NIH Publication No. 97-4051; July 1997).

The immunomodulators include, but are not limited to, the group consisting of anti-inflammatory agents, leukotriene antagonists, IL-4 muteins, soluble IL-4 receptors, immunosuppressants (such as tolerizing peptide vaccine), anti-IL-4 antibodies, IL-4 antagonists, anti-IL-5 antibodies, soluble IL-13 receptor-Fc fusion proteins, anti-IL-9 antibodies, CCR3 antagonists, CCR5 antagonists, VLA-4 inhibitors, and downregulators of IgE.

Leukotriene modifiers are often used for long-term control and prevention of symptoms in mild persistent asthma. Leukotriene modifiers function as leukotriene receptor antagonists by selectively competing for LTD-4 and LTE-4 receptors. These compounds include, but are not limited to, zafirlukast tablets and zileuton tablets. Zileuton tablets function as 5-lipoxygenase inhibitors. These drugs have been associated with the elevation of liver enzymes and some cases of reversible hepatitis and hyperbilirubinemia. Leukotrienes are biochemical mediators that are released from mast cells, eosinophils, and basophils that cause contraction of airway smooth muscle and increase vascular permeability, mucous secretions and activate inflammatory cells in the airways of patients with asthma.

Other immunomodulators include neuropeptides that have been shown to have immunomodulating properties. Functional studies have shown that substance P, for instance, can influence lymphocyte function by specific receptor-mediated mechanisms. Substance P also has been shown to modulate distinct immediate hypersensitivity responses by stimulating the generation of araclhidonic acid-derived mediators from mucosal mast cells. McGillies J et al. (1987) Fed Proc 46:196-9. Substance P is a neuropeptide first identified in 1931. Von Euler and Gaddum (1931) J Physiol (London) 72:74-87. Its amino acid sequence was reported by Chang et al. in 1971. Chang M M et al. (1971) Nature New Biol 232:86-87. The immunoregulatory activity of fragments of substance P has been studied by Siemion I Z et al. (1990) Molec Immunol 27:887-890.

Another class of compounds is the down-regulators of IgE. These compounds include peptides or other molecules with the ability to bind to the IgE receptor and thereby prevent binding of antigen-specific IgE. Another type of downregulator of IgE is a monoclonal antibody directed against the IgE receptor-binding region of the human IgE molecule. Thus, one type of downregulator of IgE is an anti-IgE antibody or antibody fragment. Anti-IgE is being developed by Genentech. One of skill in the art could prepare functionally active antibody fragments of binding peptides which have the same function. Other types of IgE downregulators are polypeptides capable of blocking the binding of the IgE antibody to the Fc receptors on the cell surfaces and displacing IgE from binding sites upon which IgE is already bound.

One problem associated with downregulators of IgE is that many molecules do not have a binding strength to the receptor corresponding to the very strong interaction between the native IgE molecule and its receptor. The molecules having this strength tend to bind irreversibly to the receptor. However, such substances are relatively toxic since they can bind covalently and block other structurally similar molecules in the body. Of interest in this context is that the a: chain of the IgE receptor belongs to a larger gene family where, e.g., several of the different IgG Fc receptors are contained. These receptors are absolutely essential for the defense of the body against, e.g., bacterial infections. Molecules activated for covalent binding are, furthermore, often relatively unstable and therefore they probably have to be administered several times a day and then in relatively high concentrations in order to make it possible to block completely the continuously renewing pool of IgE receptors on mast cells and basophilic leukocytes.

Chromolyn sodium and nedocromil are used as long-term control medications for preventing primarily asthma symptoms arising from exercise or allergic symptoms arising from allergens. These compounds are believed to block early and late reactions to allergens by interfering with chloride channel function. They also stabilize mast cell membranes and inhibit activation and release of mediators from eosinophils and epithelial cells. A four to six week period of administration is generally required to achieve a maximum benefit.

Anticholinergics are generally used for the relief of acute bronchospasm. These compounds are believed to function by competitive inhibition of muscarinic cholinergic receptors. Anticholinergics include, but are not limited to, ipratropium bromide. These compounds reverse only cholinerigically-mediated bronchospasm and do not modify any reaction to antigen. Side effects include drying of the mouth and respiratory secretions, increased wheezing in some individuals, and blurred vision if sprayed in the eyes.

In addition to standard asthma/allergy medicaments, other methods for treating asthma/allergy have been used either alone or in combination with established medicaments. One preferred, but frequently impossible, method of relieving allergies is allergen or initiator avoidance. Another method currently used for treating allergic disease involves the injection of increasing doses of allergen to induce tolerance to the allergen and to prevent further allergic reactions.

Allergen injection therapy (allergen immunotherapy) is known to reduce the severity of allergic rhinitis. This treatment has been theorized to involve the production of a different form of antibody, a protective antibody which is termed a “blocking antibody”. Cooke R A et al. (1935) Serologic Evidence of Immunity with Coexisting Sensitization in a Type of Human Allergy, Exp Med 62:733. Other attempts to treat allergy involve modifying the allergen chemically so that its ability to cause an immune response in the patient is unchanged, while its ability to cause an allergic reaction is substantially altered. These methods, however, can take several years to be effective and are associated with the risk of side effects such as anaphylactic shock.

### Formulations and Dosing

Treatment of a disease or disorder aims to reduce, ameliorate, or altogether eliminate the disease or disorder, and/or its associated symptoms, or prevent it from becoming worse. Treatment of subjects before a disease or disorder has started (i.e., prophylactic treatment) aims to reduce the risk of developing the disease or disorder. As used herein, the term “prevent” refers to the prophylactic treatment of patients who are at risk of developing a disease or disorder (resulting in a decrease in the probability that the subject will develop the disease or disorder), and to the inhibition of further development of an already established disease or disorder.

Different doses may be necessary for treatment of a subject, depending on activity of the compound, manner of administration, purpose of the treatment (i.e., prophylactic or therapeutic), nature and severity of the disease or disorder, age and body weight of the subject. The administration of a given dose can be carried out both by single administration in the form of an individual dose unit or else by several dose units. Multiple administration of doses at specific intervals of weeks or months apart is usual for boosting antigen-specific immune responses.

Combined with the teachings provided herein, by choosing among the various active compounds and weighing factors such as potency, relative bioavailability, patient body weight, severity of adverse side-effects, and mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular therapeutic agent being administered (e.g., in the case of an immunostimulatory nucleic acid, the type of nucleic acid, i.e., a CpG nucleic acid, the number of unmethylated CpG motifs or their location in the nucleic acid, the degree of modification of the backbone to the oligonucleotide, etc.), the size of the subject, or the severity of the disease or condition. One of ordinary skill in the art can empirically determine the effective amount of a particular conjugate and/or other therapeutic agent without necessitating undue experimentation.

Subject doses of the compounds described herein typically range from about 0.1 g to 10,000 mg, more typically from about 1 μg/day to 8000 mg, and most typically from about 10 μg to 100 μg. Stated in terms of subject body weight, typical dosages range from about 0.1 μg to 20 mg/kg/day, more typically from about 1 to 10 mg/kg/day, and most typically from about 1 to 5 mg/kg/day.

The pharmaceutical compositions containing conjugates of the invention and/or other compounds can be administered by any suitable route for administering medications. A variety of administration routes are available. The particular mode selected will depend, of course, upon the particular agent or agents selected, the particular condition being treated, and the dosage required for therapeutic efficacy. The methods of this invention, generally speaking, may be practiced using any mode of administration that is medically acceptable, meaning any mode that produces an effective response without causing clinically unacceptable adverse effects. Preferred modes of administration are discussed herein. For use in therapy, an effective amount of the conjugate and/or other therapeutic agent can be administered to a subject by any mode that delivers the agent to the desired surface, e.g., mucosal, systemic.

Administering the pharmaceutical composition of the present invention may be accomplished by any means known to the skilled artisan. Routes of administration include but are not limited to oral, mucosal, parenteral, intravenous, intramuscular, intranasal, sublingual, intratracheal, inhalation, intradermal, subcutaneous (s.c.), ocular, vaginal, and rectal. For the treatment or prevention of asthma or allergy, such compounds may be inhaled, ingested or administered by systemic routes. Systemic routes include oral and parenteral. Inhaled medications are preferred in some embodiments because of the direct delivery to the lung, the site of inflammation, primarily in asthmatic patients. Several types of devices are regularly used for administration by inhalation. These types of devices include metered dose inhalers (MDI), breath-actuated MDI, dry powder inhaler (DPI), spacer/holding chambers in combination with MDI, and nebulizers.

The therapeutic agents of the invention may be delivered to a particular tissue, cell type, or to the immune system, or both, with the aid of a vector. In its broadest sense, a “vector” is any vehicle capable of facilitating the transfer of the compositions to the target cells. The vector generally transports the conjugate, immunostimulatory nucleic acid, antibody, antigen, and/or disorder-specific medicament to the target cells with reduced degradation relative to the extent of degradation that would result in the absence of the vector.

In general, vectors useful in the invention are divided into two classes: biological vectors and chemical/physical vectors. Biological vectors and chemical/physical vectors are useful in the delivery and/or uptake of therapeutic agents of the invention.

Most biological vectors are used for delivery of nucleic acids and this would be most appropriate in the delivery of therapeutic agents that are or that include immunostimulatory nucleic acids.

In addition to the biological vectors discussed herein, chemical/physical vectors may be used to deliver therapeutic agents including immunostimulatory nucleic acids, antibodies, antigens, and disorder-specific medicaments. As used herein, a “chemical/physical vector” refers to a natural or synthetic molecule, other than those derived from bacteriological or viral sources, capable of delivering the conjugate and/or other medicament.

In one embodiment a chemical/physical vector of the invention is a colloidal dispersion system. Colloidal dispersion systems include lipid-based systems including oil-in-water emulsions, micelles, mixed micelles, and liposomes. In one embodiment a colloidal system of the invention is a liposome. Liposomes are artificial membrane vessels which are useful as a delivery vector in vivo or in vitro. It has been shown that large unilamellar vesicles (LUVs), which range in size from 0.2-4.0 μm, can encapsulate large macromolecules. RNA, DNA and intact virions can be encapsulated within the aqueous interior and be delivered to cells in a biologically active form. Fraley R et al. (1981) Trends Biochem Sci 6:77.

Liposomes may be targeted to a particular tissue by coupling the liposome to a specific ligand such as a monoclonal antibody, sugar, glycolipid, or protein. Ligands which may be useful for targeting a liposome to an immune cell include, but are not limited to: intact or fragments of molecules which interact with immune cell specific receptors and molecules, such as antibodies, which interact with the cell surface markers of immune cells. Such ligands may easily be identified by binding assays well known to those of skill in the art. In still other embodiments, the liposome may be targeted to the cancer by coupling it to a one of the immunotherapeutic antibodies discussed earlier. Additionally, the vector may be coupled to a nuclear targeting peptide, which will direct the vector to the nucleus of the host cell.

Lipid formulations for transfection are commercially available from QIAGEN, for example, as EFFECTENE™ (a non-liposomal lipid with a special DNA condensing enhancer) and SUPERFECT™ (a novel acting dendrimeric technology).

Liposomes are commercially available from Gibco BRL, for example, as LIPOFECTIN™ and LIPOFECTACE™, which are formed of cationic lipids such as N-[1-(2,3 dioleyloxy)-propyl]-N,N, N-trimethylammonium chloride (DOTMA) and dimethyl dioctadecylammonium bromide (DDAB). Methods for making liposomes are well known in the art and have been described in many publications. Liposomes also have been reviewed by Gregoriadis G (1985) Trends Biotechnol 3:235-241.

In one embodiment, the vehicle is a biocompatible microparticle or implant that is suitable for implantation or administration to the mammalian recipient. Exemplary bioerodible implants that are useful in accordance with this method are described in published international patent application WO95/24929, entitled “Polymeric Gene Delivery System”. This published application describes a biocompatible, preferably biodegradable polymeric matrix for containing an exogenous gene under the control of an appropriate promoter. The polymeric matrix can be used to achieve sustained release of the therapeutic agent in the subject.

The polymeric matrix preferably is in the form of a microparticle such as a microsphere (wherein the conjugate and/or the other therapeutic agent is dispersed throughout a solid polymeric matrix) or a microcapsule (wherein the conjugate and/or the other therapeutic agent is stored in the core of a polymeric shell). Other forms of the polymeric matrix for containing the therapeutic agent include films, coatings, gels, implants, and stents. The size and composition of the polymeric matrix device is selected to result in favorable release kinetics in the tissue into which the matrix is introduced. The size of the polymeric matrix further is selected according to the method of delivery which is to be used, typically injection into a tissue or administration of a suspension by aerosol into the nasal and/or pulmonary areas. Preferably when an aerosol route is used the polymeric matrix and the conjugate and/or the other therapeutic agent are encompassed in a surfactant vehicle. The polymeric matrix composition can be selected to have both favorable degradation rates and also to be formed of a material which is bioadhesive, to further increase the effectiveness of transfer when the matrix is administered to a nasal and/or pulmonary surface that has sustained an injury. The matrix composition also can be selected not to degrade, but rather, to release by diffusion over an extended period of time. In some preferred embodiments, the conjugate is administered to the subject via an implant while the other therapeutic agent is administered acutely. Biocompatible microspheres that are suitable for delivery, such as oral or mucosal delivery, are disclosed in Chickering et al. (1996) Biotech Bioeng 52:96-101 and Mathiowitz E et al. (1997) Nature 386:410-414 and published international patent application WO97/03702.

Both non-biodegradable and biodegradable polymeric matrices can be used to deliver the conjugate and/or the other therapeutic agent to the subject. Biodegradable matrices are preferred. Such polymers may be natural or synthetic polymers. The polymer is selected based on the period of time over which release is desired, generally in the order of a few hours to a year or longer. Typically, release over a period ranging from between a few hours and three to twelve months is most desirable, particularly for nucleic acid agents. The polymer optionally is in the form of a hydrogel that can absorb up to about 90% of its weight in water and further, optionally is cross-linked with multi-valent ions or other polymers.

Bioadhesive polymers of particular interest include bioerodible hydrogels described by Sawhney A S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

If the therapeutic agent is a nucleic acid, the use of compaction agents may also be desirable. Compaction agents also can be used alone, or in combination with, a biological or chemical/physical vector. A “compaction agent”, as used herein, refers to an agent, such as a histone, that neutralizes the negative charges on the nucleic acid and thereby permits compaction of the nucleic acid into a fine granule. Compaction of the nucleic acid facilitates the uptake of the nucleic acid by the target cell. The compaction agents can be used alone, i.e., to deliver a nucleic acid in a form that is more efficiently taken up by the cell or, more preferably, in combination with one or more of the above-described vectors.

Other exemplary compositions that can be used to facilitate uptake of a nucleic acid include calcium phosphate and other chemical mediators of intracellular transport, microinjection compositions, electroporation and homologous recombination compositions (e.g., for integrating a nucleic acid into a preselected location within the target cell chromosome).

The compounds may be administered alone (e.g., in saline or buffer) or using any delivery vectors known in the art. For instance the following delivery vehicles have been described: cochleates (Gould-Fogerite et al., 1994, 1996); Emulsomes (Vancott et al., 1998, Lowell et al., 1997); ISCOMs (Mowat et al., 1993, Carlsson et al., 1991, Hu et., 1998, Morein et al., 1999); liposomes (Childers et al., 1999, Michalek et al., 1989, 1992, de Haan 1995a, 1995b); live bacterial vectors (e.g., Salmonella, Escherichia coli, Bacillus Calmette-Guérin, Shigella, Lactobacillus) (Hone et al., 1996, Pouwels et al., 1998, Chatfield et al., 1993, Stover et al., 1991, Nugent et al., 1998); live viral vectors (e.g., Vaccinia, adenovirus, Herpes simplex) (Gallichan et al., 1993, 1995, Moss et al., 1996, Nugent et al., 1998, Flexner et al., 1988, Morrow et al., 1999); microspheres (Gupta et al., 1998, Jones et al., 1996, Maloy et al., 1994, Moore et al., 1995, O'Hagan et al., 1994, Eldridge et al., 1989); nucleic acid vaccines (Fynan et al., 1993, Kuklin et al., 1997, Sasaki et al., 1998, Okada et al., 1997, Ishii et al., 1997); polymers (e.g., carboxymethylcellulose, chitosan) (Hamajima et al., 1998, Jabbal-Gill et al., 1998); polymer rings (Wyatt et al., 1998); proteosomes (Vancott et al., 1998, Lowell et al., 1988, 1996, 1997); sodium fluoride (Hashi et al., 1998); transgenic plants (Tacket et al., 1998, Mason et al., 1998, Haq et al., 1995); virosomes (Gluck et al., 1992, Mengiardi et al., 1995, Cryz et al., 1998); and, virus-like particles (Jiang et al., 1999, Leibl et al., 1998).

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

Components of the pharmaceutical compositions also are capable of being comingled with the compounds of the present invention, and with each other, in a manner such that there is no interaction which would substantially impair the desired pharmaceutical efficiency.

For oral administration, the compounds (I.e., conjugates, nucleic acids, antigens, antibodies, and other therapeutic agents) can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long-acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R (1990) Science 249:1527-33, which is incorporated herein by reference.

The conjugates and optionally other therapeutics and/or antigens may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% W/v).

The compositions may conveniently be presented in unit dosage form and may be prepared by any of the methods well known in the art of pharmacy. All methods include the step of bringing the compounds into association with a carrier which constitutes one or more accessory ingredients. In general, the compositions are prepared by uniformly and intimately bringing the compounds into association with a liquid carrier, a finely divided solid carrier, or both, and then, if necessary, shaping the product. Liquid dose units are vials or ampoules. Solid dose units are tablets, capsules and suppositories.

Other delivery systems can include time-release, delayed release or sustained release delivery systems. Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-, di-, and tri-glycerides; hydrogel release systems; silastic systems; peptide-based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

### EXAMPLES

### Example 1

### Conjugation of A Basic Oligonucleotide to Ovalbumin and Analysis of Conjugates

Ovalbumin (OVA) is incubated with the crosslinker sulfo-maleimidobenzoyl-N-hydroxysuccinimide ester (S-MBS; Pierce, Germany) in 50 mM EDTA-PBS buffer pH 7.0 at a molar ratio of 1:10 for 1 h at room temperature. Sulfhydril-modified 20-mer phosphorothioate abasic oligodeoxynucleotide (abasic ODN) is reduced in a 50 mM solution of 1,4-dithiothreitol-PBS. Subsequently unbound S-MBS and 1,4-dithiothreitol are removed by chromatography on a Biorade P-6 gel column (Biorade, Germany). The activated abasic ODN is incubated with the linker-modified ovalbumin at a molar ratio of 5:1 for 2.5 h at room temperature and thereafter L-cysteine is added to quench reactive S-MBS. Free abasic ODN is removed by chromatography on a Superdex 75HR column (Amersham Biosciences, Germany). Purified conjugates are analyzed on a 6-20% gradient SDS-PAGE and silverstained. To determine ratio of bound abasic ODN on ovalbumin a 4-15% gradient non-denaturing, non-reducing PAGE is run and silverstained or visualized using ethidium bromide staining. Protein concentration is determined by the Lowry method (Pierce, Germany).

### Example 2

### Uptake Analysis

FITC label can be used as a tracking marker. 5′FITC-labeled oligonucleotides are synthesized and then conjugated as in Example 1. To examine the uptake of FITC-labeled conjugate in vivo, 0.5 μg protein (2.8 pmole abasic ODN) is injected s.c. into the foot pads of naïve 8-12 week-old C57BL/6 mice (Harlan Winkelmann GmbH, Germany). Lymph nodes are aseptically removed and digested for 1 h at 37° C. in 5% CO₂ atmosphere using collagenase Type Ia (Sigma, Germany). Single-cell suspensions are prepared and clumps removed using a 100 μm pore size filter (Falcon, Germany). Cells are stained with magnetic beads coated with anti-CD11c monoclonal antibody (clone HL3, PharMingen, Germany) and separated into CD11c⁺ and CD11c⁻ cell fractions using MiniMACS and MS⁺ separation columns according to the manufacturer's instructions (Miltenyi Biotech, Germany).

To examine the uptake of FITC-labeled conjugates in vitro, bone marrow-derived DC are exposed to FITC-labeled ovalbumin (1 h at 37° C.), washed with ice-cold 2% FCS-PBS containing 2 mM EDTA and stained with anti-CD11c-APC. To examine the ability of “third party” ODN to block uptake of FITC-labeled conjugates, cells of the macrophage line ANA-1 or immature DC are incubated with OVA-FITC alone, mixed or conjugated with 20-mer phosphorothioate abasic ODN for 1 h at 37° C. Increasing concentrations of free phosphorothioate abasic ODN, CpG ODN 1668 (5′-TCCATGACGTTCCTGATGCT-3′; SEQ ID NO: 1), GpC ODN 1720 (5′-TCCATGAGCTTCCTGATGCT-3′; SEQ ID NO:2), or CpG ODN 1668 modified with a poly-G tail (5′-TCCATGACGTTCCTGGGGGG-3′; SEQ ID NO:3) are added. To ensure intracellular uptake, surface staining of OVA-FITC is quenched by adding 50 μg/ml trypan blue.

To analyze DC activation, Flt3-ligand cultured bone marrow-derived DC are incubated with 17.6 μg/ml OVA alone, mixed, or conjugated to 1 μM abasic ODN. Cells are cultured for 24 h, then washed and stained with APC-labeled anti-CD11c, FITC-labeled anti-CD40, or FITC-labeled anti-CD86. FACS analysis is performed on a FACSCaliber flow cytometer (Becton Dickinson, Germany) acquiring at least 30,000 events per sample. FACS data is analyzed using CellQuest software.

### Example 3

### Presentation Assay

Presentation of OVA peptide SIINFEKL (OVA peptide 257-264; SEQ ID NO:4) ex vivo is assayed as previously described by measuring induction of lacZ activity in the SIINFEKL/K^{b}-specific T cell hybridoma B3Z. Vabulas R M et al. (2000) J Immunol. 164:2372-8; Specht J M et al. (1997) J Exp Med 186:1213-21. To this B3Z cells and positively selected CD11c⁺ lymph node cells are co-cultured. Twelve hours after antigen injection draining lymph nodes are harvested and dissociated lymph node cells are exposed to magnetic beads coated with anti-CD11c monoclonal antibody. For separation into CD11c⁺ and CD11c⁻ subpopulations, MiniMACS and MS⁺ separation columns are used according to the manufacturer's instructions (Miltenyi Biotech, Germany). Defined numbers of fractionated cells are incubated with B3Z at 37° C. overnight. Cells are then fixed with 0.5% glutaraldehyde for 10 min and incubated with X-Gal solution at 37° C. for 4-8 h. Blue cells are counted under the microscope.

To evaluate OVA peptide presentation in vitro, 2×10⁵ Flt3-ligand cultured cells are incubated with indicated substances for 5 h at 37° C. Plates are washed and 5×10³ B3Z cells are added to each well. After additional incubation overnight at 37° C., cells are lysed by addition of 100 ml Z-buffer (100 mM 2-mercaptoethanol, 9 mM MgCl₂, 0.125% Nonidet P-40, 0.15 mM chlorophenol red P-galactoside (Calbiochem, San Diego, Calif.) in PBS) and after 24 h absorption of individual cells is read using a 96-well Emex plate reader (Molecular Devices, Sunnyvale, Calif.) at 570 nm, with 650 nm as reference wavelength.

### Example 4

### In Vitro Uptake of A basic Oligonucleotides and CpG-ODN

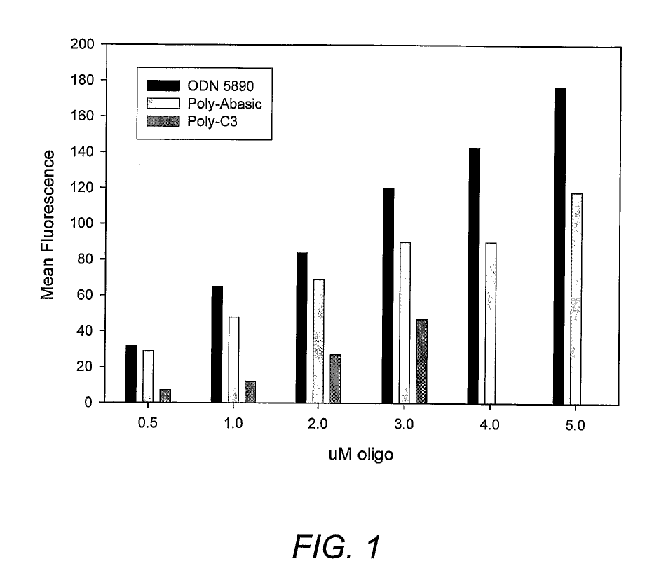

The 20-mer ODN 5890 (TCCATGACGTTTTTGATGTT; SEQ ID NO:5), a 20-mer poly-abasic (i.e., poly-D) or a 20-mer poly-C3 were synthesized with a fluorescence tag Cy₃. The murine macrophage cell line, RAW 264.7 (American Type Culture Collection, Manassas, Va.), was incubated with various concentrations of test oligomer (0.5 to 5.0 μM) for 1 hr at 37° C. The cells were then washed and FACS analyzed for oligomer uptake by monitoring mean fluorescence. Results are shown in FIG. 1. Although the ODN was up taken to a greater extent than either abasic oligonucleotide, poly-D was up taken between 75-80% as efficiently, while poly-C3 was taken up roughly 35% as efficiently. No data is shown for poly-C3 at concentrations of 4.0 or 5.0 μM.

### Example 5

### TLR9Signaling Induction by CpG Motif in Various Contexts

Following published methods, HEK 293 cells were stably transfected with a murine TLR9 expression vector and a six-fold NFκB-luciferase reporter plasmid. Cells were plated on 96-well plates at 1.5×10⁴ cells/well and allowed to attach overnight. The cells were then treated for sixteen hours with individual test compounds listed below at concentrations ranging between 10⁻⁹ M and 10⁻⁵ M.

Test agents were as follows: ODN 20321 (GACGTT); ODN 5890 (SEQ ID NO:5); 20307 (DDDDDGACGTTDDDDDDDDD, where each D represents an abasic deoxyribonucleotide unit); and 20566 (JJJJJGACGTTJJJJJJJJJ, where each J represents a C3 spacer derived from propane-1,3-diol).

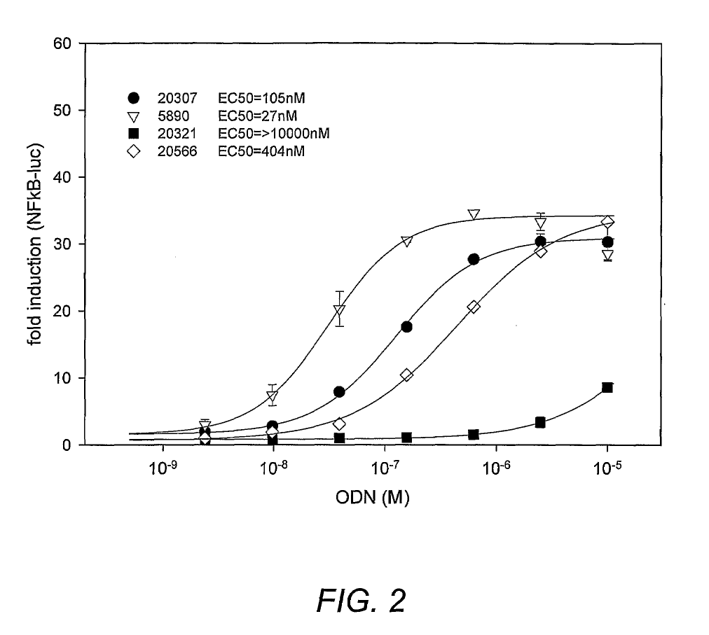

Each data point was done in duplicate. After 16 h stimulation the supernatant was removed and the cells were treated with lysis buffer and stored at −80° C. until luciferase measurement. Values are given as fold NFκB activation compared with non-stimulated cells. Results are shown in FIG. 2.

As shown in FIG. 2, ODN 5890 and both oligonucleotides 20307 and 20566 induced significantly more TLR9 signaling in this assay than did hexamer CpG motif alone (20321). EC₅₀ values for the various agents were as follows: 20321, >10,000 nM; 20566, 404 nM; 20307, 105 nM; and 5890, 27 mM.

### EQUIVALENTS

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

All references, patents and patent publications that are recited in this application are incorporated in their entirety herein by reference.

We claim:

1. A composition comprising a conjugate of an abasic oligonucleotide 10-40 units long and a therapeutic agent.

2. The composition of claim 1, wherein the abasic oligonucleotide is a homopolymer of abasic deoxyribonucleotides.

3. The composition of claim 1, wherein the abasic oligonucleotide is a homopolymer of abasic ribonucleotides.

4. The composition of claim 1, wherein the abasic oligonucleotide is a heteropolymer of abasic ribonucleotides and abasic deoxyribonucleotides.

5. The composition of claim 1, wherein the abasic oligonucleotide is a homopolymer of C3 spacers derived from propane-1,3-diol.

6. The composition of claim 1, wherein the units are linked by phosphodiester linkages.

7. The composition of claim 1, wherein the units are linked by phosphorothioate linkages.

8. The composition of claim 1, wherein the therapeutic agent is an antigen.

9. The composition of claim 1, wherein the therapeutic agent is an immunostimulatory nucleic acid molecule.

10. The composition of claim 1, wherein the therapeutic agent is a CpG oligonucleotide.

11. The composition of claim 1, wherein the therapeutic agent is a small molecule.

12. The composition of claim 11, wherein the small molecule is a Toll-like receptor (TLR) signaling agonist.

13. The composition of claim 11, wherein the small molecule is a Toll-like receptor (TLR) signaling antagonist.

14. The composition of claim 1, wherein the therapeutic agent is a plurality of identical therapeutic agents.

15. The composition of claim 1, wherein the therapeutic agent comprises a plurality of non-identical therapeutic agents.

16. The composition of claim 1, wherein the abasic oligonucleotide and the therapeutic agent are covalently coupled.

17. The composition of claim 16, wherein the abasic oligonucleotide comprises a 5′ end and a 3′ end and the therapeutic agent is covalently coupled to the 3′ end of the abasic oligonucleotide.

18. The composition of claim 16, wherein the abasic oligonucleotide comprises a 5′ end and a 3′ end and the therapeutic agent is covalently coupled to the 5′ end of the abasic oligonucleotide.

19. The composition of claim 1, wherein the abasic oligonucleotide and the therapeutic agent are covalently coupled through a linker.

20. The composition of claim 19, wherein the linker is susceptible to cleavage by an enzyme.

21. The composition of claim 1, wherein the abasic oligonucleotide is at least 20 units long.

22. The composition of claim 1, wherein the abasic oligonucleotide is 20 units long.

23. The composition of claim 1, further comprising a pharmaceutically acceptable carrier.

24-29. (canceled)

30. A vaccine comprising an abasic oligonucleotide 10-40 units long covalently linked to an antigen.

31-33. (canceled)

34. A method of increasing antigen uptake by an antigen-presenting cell (APC), comprising
contacting an APC with a composition comprising a conjugate of an abasic oligonucleotide 10-40 units long and an antigen, in an effective amount to permit antigen uptake by the APC, wherein for a given amount of the antigen, an amount of the antigen taken up by the APC is greater when the APC is contacted with the conjugate than when the APC is contacted with the antigen alone.

35-39. (canceled)

40. A method of vaccinating a subject, comprising
administering to a subject a composition comprising a conjugate of an abasic oligonucleotide 10-40 units long and an antigen, in an effective amount to induce an antigen-specific immune response to the antigen in the subject.

41. A method of increasing delivery of a Toll-like receptor (TLR) signaling agonist to a TLR, comprising
contacting a cell comprising a TLR with a composition comprising a conjugate of an abasic oligonucleotide 10-40 units long and a TLR signaling agonist specific for the TLR, in an effective amount to deliver the TLR signaling agonist to the TLR, wherein for a given amount of the TLR signaling agonist, an amount of the TLR signaling agonist delivered to the TLR is greater when the cell is contacted with the conjugate than when the cell is contacted with the TLR signaling agonist alone.

42-49. (canceled)

50. A composition comprising a conjugate of at least one abasic oligonucleotide and an immunostimulatory nucleic acid molecule, wherein the conjugate includes at least 4 abasic units and the immunostimulatory nucleic acid includes at least 6 nucleotides, wherein the conjugate is 10-40 units and nucleotides long.

51-53. (canceled)
